(19) United States
(12) Patent Application Publication
(10) Pub. No.: US 2017/0128483 A1
(43) Pub. Date: May 11, 2017

(54) HOST DEPENDENCY FACTORS AS TARGETS FOR ANTIVIRAL THERAPY

(71) Applicant: Ruprecht-Karls-Universitat Heidelberg, Heidelberg (DE)

(72) Inventors: Wolfgang FISCHL, Vienna (AT); Eliana ACOSTA, Heidelberg (DE); Alessia RUGGIERI, Heidelberg (DE); Ralf BARTENSCHLAGER, Schriesheim (DE)

(21) Appl. No.: 15/127,277

(22) PCT Filed: Mar. 24, 2015
(86) PCT No.: PCT/EP2015/056228

§ 371 Date: Sep. 19, 2016

(30) Foreign Application Priority Data
Mar. 25, 2014 (EP) 14161494.1

(51) Int. Cl. A61K 31/7076; A61K 31/166; G01N 33/50; A61K 31/427; A61K 31/155; A61K 31/4709; A61K 45/06; A61K 31/381

(52) U.S. Cl. CPC A61K 31/7076; A61K 45/06; A61K 31/166; A61K 31/381; A61K 31/427; A61K 31/155; A61K 31/4709; G01N 33/502; G01N 2500/10

(57) ABSTRACT

The present invention relates to inhibitor(s) or antagonist(s) of PADI4 (peptidyl arginine deiminase, type IV), PPARδ (peroxisome proliferator-activated receptor delta), GCKR (glucokinase regulatory protein), and/or P2X4R (purinergic receptor P2X, ligand-gated ion channel 4) for the use as anti-viral agent(s) as well as for the use in the prevention and/or treatment of infection(s) with virus(es) of the Flaviviridae family, such as Dengue virus and hepatitis C virus (HCV). The present invention further relates to pharmaceutical compositions or kits comprising said inhibitor(s)/antagonist(s) and methods of preventing and/or treating infection(s) with virus(es) of the Flaviviridae family. The present invention further relates to methods of screening for antiviral agent(s). The present invention relates to PADI4 (peptidyl arginine deiminase, type IV), PPARδ (peroxisome proliferator-activated receptor delta), GCKR (glucokinase regulatory protein), and/or P2X4R (purinergic receptor P2X, ligand-gated ion channel 4) for the use in diagnosis, prevention and/or treatment of infection(s) with virus(es) of the Flaviviridae family, preferably as targets for antiviral treatment or as screening targets.

The present invention relates to inhibitor(s) or antagonist(s) of PADI4 (peptidyl arginine deiminase, type IV), PPARδ (peroxisome proliferator-activated receptor delta), GCKR (glucokinase regulatory protein), and/or P2X4R (purinergic receptor P2X, ligand-gated ion channel 4) for the use as anti-viral agent(s) as well as for the use in the prevention and/or treatment of infection(s) with virus(es) of the Flaviviridae family, such as Dengue virus and hepatitis C virus (HCV). The present invention further relates to pharmaceutical compositions or kits comprising said inhibitor(s)/antagonist(s) and methods of preventing and/or treating infection(s) with virus(es) of the Flaviviridae family. The present invention further relates to methods of screening for antiviral agent(s). The present invention relates to PADI4 (peptidyl arginine deiminase, type IV), PPARδ (peroxisome proliferator-activated receptor delta), GCKR (glucokinase regulatory protein), and/or P2X4R (purinergic receptor P2X, ligand-gated ion channel 4) for the use in diagnosis, prevention and/or treatment of infection(s) with virus(es) of the Flaviviridae family, preferably as targets for antiviral treatment or as screening targets.

### BACKGROUND OF THE INVENTION

Dengue is the most prevalent mosquito-borne viral disease, causing an estimated 390 million infections annually (Bhatt et al. 2013). In the majority of infections, patients remain asymptomatic or develop the self-limited dengue fever (DF), but in certain cases individuals develop more severe symptoms and the disease progresses to the potentially fatal forms dengue hemorrhagic fever (DHF) and dengue shock syndrome (DSS) (Gubler 1998, Halstead 2007). Despite considerable effort devoted to the development of a DENV vaccine or antiviral drugs, neither is available yet.

Dengue virus (DENV) is a member of the family Flaviviridae, which contains other notable human pathogens such as Yellow fever virus, West Nile virus, Japanese encephalitis virus, Tick-borne encephalitis virus and Hepatitis C virus (HCV). The four antigenically distinct DENV species (DENV-1 to DENV-4), taxonomically ranked as serotypes, are currently co-circulating in tropical and subtropical regions of the world where approximately 2.5 billion people are at risk of infection (Halstead 2007). The viruses are transmitted to humans through the bite of an infected mosquito vector (mainly Aedes aegypti). It is speculated that subcutaneously infected virus encounters and infects tissue-resident dendritic cells (DCs) and macrophages (St John et al, 2013), which eventually migrate to lymph nodes where recruited macrophages and monocytes become additional targets, amplifying the infection further (Martina, et al 2009). Although DCs, monocytes, and macrophages are considered as the major sites of virus replication in humans (Limon-Flores et al 2005, Wu et al 2000), the virus can also be detected in various other tissues, including spleen, kidneys, lungs, and the liver (Jessie et al. 2004, Seneviratne et al 2006).

Due to their limited coding capacity, viruses exploit components and pathways of the host cell to assure productive replication. However, the host cell tries to eliminate or counteract the invader by mounting various antiviral responses to clear the infection. In the case of DENV, this complex host-pathogen interplay is largely unexplored. The discovery and characterization of host cell factors involved in virus replication not only sheds light into cell biological processes and pathways, but also represents an attractive strategy for novel antiviral approaches. The error-prone viral polymerases allow a fast evolution and adaptation, enabling viruses to quickly develop resistance to antivirals targeting viral proteins. In contrast, host factors are not subject to such a rapid changes and therefore targeting a cellular protein is thought to reduce the risk for the emergence of drug-resistant virus variants.

Thus, there is a need in the art for providing improved means and methods for the prevention and/or treatment as well as the diagnosis of infections with virus(es) of the Flaviviridae family, such as DENV.

There is also a need in the art for providing novel anti-viral agent(s) and target(s).

### SUMMARY OF THE INVENTION

According to the present invention this object is solved by providing inhibitor(s) or antagonist(s) of PADI4 (peptidyl arginine deiminase, type IV), PPARδ (peroxisome proliferator-activated receptor delta), GCKR (glucokinase regulatory protein), and/or P2X4R (purinergic receptor P2X, ligand-gated ion channel 4) for the use as anti-viral agent(s).

According to the present invention this object is solved by providing inhibitor(s) or antagonist(s) of PADI4 (peptidyl arginine deiminase, type IV), PPARδ (peroxisome proliferator-activated receptor delta), GCKR (glucokinase regulatory protein), and/or P2X4R (purinergic receptor P2X, ligand-gated ion channel 4) for the use in the prevention and/or treatment of infection(s) with virus(es) of the Flaviviridae family.

According to the present invention this object is solved by providing a pharmaceutical composition comprising at least one inhibitor or antagonist of PADI4, PPARδ, GCKR and/or P2X4R according to the invention; optionally, a pharmaceutical excipient, and/or optionally, a further antiviral agent.

According to the present invention this object is solved by providing the pharmaceutical composition according to the invention for use in preventing and/or treating of infection(s) with virus(es) of the Flaviviridae family.

According to the present invention this object is solved by a method for the prevention and/or treatment of infection(s) with virus(es) of the Flaviviridae family, comprising
administering to a patient at least one inhibitor or antagonist of PADI4, PPARδ, GCKR and/or P2X4R according to the invention or a pharmaceutical composition of the invention.

According to the present invention this object is solved by a method of screening for antiviral agent(s), comprising

(a) adding a compound to be screened to a PADI4, PPARδ, GCKR and/or P2X4R test system, preferably a cell line expressing PADI4, PPARδ, GCKR and/or P2X4R;

(b) infecting said test system with a virus of the Flaviviridae family in the presence of said compound to be screened;

(c) removing the viral inocula and adding said compound to be screened;

(d) quantifying virus production; and

(e) comparing virus production in step (d) with the virus production in the absence of the candidate compound, wherein a difference between the measured virus productions indicates that the candidate compound is a modulator of PADI4, PPARδ, GCKR and/or P2X4R (wherein a decrease in the measured virus production in step (d) indicates that the candidate compound is an inhibitor or antagonist of PADI4, PPARδ, GCKR and/or P2X4R and wherein an increase in the measured virus production in step (d) indicates that the candidate compound is an agonist of PADI4, PPARδ, GCKR and/or P2X4R).

According to the present invention this object is solved by a method of screening for antiviral agent(s), comprising

(a) infecting a PADI4, PPARδ, GCKR and/or P2X4R test system, preferably a cell line expressing PADI4, PPARδ, GCKR and/or P2X4R, with a virus of the Flaviviridae family;

(b) removing the viral inocula and adding a compound to be screened;

(c) quantifying virus production; and

(d) comparing virus production in step (c) with the virus production in the absence of the candidate compound, wherein a difference between the measured virus productions indicates that the candidate compound is a modulator of PADI4, PPARδ, GCKR and/or P2X4R (wherein a decrease in the measured virus production in step (c) indicates that the candidate compound is an inhibitor or antagonist of PADI4, PPARδ, GCKR and/or P2X4R and wherein an increase in the measured virus production in step (c) indicates that the candidate compound is an agonist of PADI4, PPARδ, GCKR and/or P2X4R).

According to the present invention this object is solved by providing PADI4 (peptidyl arginine deiminase, type IV), PPARδ (peroxisome proliferator-activated receptor delta), GCKR (glucokinase regulatory protein), and/or P2X4R (purinergic receptor P2X, ligand-gated ion channel 4) for the use in diagnosing, preventing and/or treating infection(s) with virus(es) of the Flaviviridae family.

According to the present invention this object is solved by providing a kit for diagnosing, preventing and/or treating infection(s) with virus(es) of the Flaviviridae family, comprising
at least one inhibitor or antagonist of PADI4, PPARδ, GCKR and/or P2X4R according to the invention,
and/or at least one of PADI4, PPARδ, GCKR and/or P2X4R,
optionally, suitable substrates of PADI4, PPARδ, GCKR and/or P2X4R for in vitro enzymatic assays,
optionally, a PADI4, PPARδ, GCKR and/or P2X4R test system, such as a cell line expressing PADI4, PPARδ, GCKR and/or P2X4R;
optionally, excipient(s) and further compounds.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

### Anti-Viral Agent(s)

As described above, the present invention provides an inhibitor or antagonist of PADI4 (peptidyl arginine deiminase, type IV), PPARδ (peroxisome proliferator-activated receptor delta), GCKR (glucokinase regulatory protein), and/or P2X4R (purinergic receptor P2X, ligand-gated ion channel 4) for the use as anti-viral agent, preferably as agent against virus(es) of the Flaviviridae family.

As described above, the present invention provides an inhibitor or antagonist of PADI4 (peptidyl arginine deiminase, type IV), PPARδ (peroxisome proliferator-activated receptor delta), GCKR (glucokinase regulatory protein), and/or P2X4R (purinergic receptor P2X, ligand-gated ion channel 4) for the use in preventing and/or treating of infection(s) with virus(es) of the Flaviviridae family.

Preferably, the virus(es) of the Flaviviridae family are members of the genus Flavivirus and/or the genus Hepacivirus.

Preferably, the virus(es) of the Flaviviridae family are Dengue virus (DENV), Hepatitis C virus (HCV), Yellow fever virus, West Nile virus, Japanese encephalitis virus and Tick-borne encephalitis virus,

wherein the Dengue virus preferably comprises the four serotypes DENV-1, DENV-2, DENV-3 and DENV-4.

The Flaviviridae are a family of enveloped RNA viruses. The family gets its name from Yellow Fever virus, a type virus of Flaviviridae; flavus means yellow in Latin. Flaviviridae have monopartite, linear, single-stranded RNA genomes of positive polarity, 9.6 to 12.3 kilobase in length. The 5′-termini of flaviviruses carry a methylated nucleotide cap, while RNA genomes of other members of this family are uncapped and contain an internal ribosome entry site. Virus particles are enveloped and spherical, about 40-80 nm in diameter. Flaviviridae, more specifically members of the genus Flavivirus, infect a wide range of vertebrates and are spread through arthropods, mainly ticks and mosquitoes whereas members of the genus Hepacivirus, including HCV, are transmitted parenterally (through blood) as well as sexually and vertically (from mother to child).

The Flaviviridae family includes the following genera:
Genus Flavivirus (type species Yellow fever virus, others include West Nile virus and Dengue virus)—contains 67 identified human and animal viruses
Genus Hepacivirus (type species Hepatitis C virus, also includes GB virus B)
Genus Pegivirus (includes GB virus A, GB virus C, and GB virus D)
Genus Pestivirus (type species bovine viral diarrhea virus, others include classical swine fever virus or border disease virus)—contains viruses infecting non-human mammals

According to the invention, “treating” of infection(s) with virus(es) of the Flaviviridae family includes
alleviating the infection, including decreasing virus replication and/or
reducing viral load in serum or infected tissues and/or
reducing amounts of inflammatory cytokines and/or
reducing pathogenicity of the virus and/or and/or
reducing disease symptoms.

In one embodiment, the inhibitor or antagonist is selected from
N-α-benzoyl-N5-(2-chloro-1-iminoethyl)-L-ornithine amide (Cl-amidine);
3-(((2-Methoxy-4-(phenylamino)phenyl)amino)sulfonyl)-2-thiophene-carboxylic acid methyl ester (GSK0660);
2-Amino-5-(4-methyl-4H-(1,2,4)-triazole-3-yl-sulfanyl-N-(4-methyl-thiazole-2-yl)benzamide (CpdA);
and/or
2′,3′,-O-(2,4,6-Trinitrophenyl) adenosine 5 ′-triphosphate monolithium-trisodium salt (TNP-ATP).

In one embodiment, the inhibitor or antagonist is selected from
N-α-benzoyl-N5-(2-chloro-1-iminoethyl)-L-ornithine amide (Cl-amidine);
3-(((2-Methoxy-4-(phenylamino)phenyl)amino)sulfonyl)-2-thiophene-carboxylic acid methyl ester (GSK0660);
2-Amino-5-(4-methyl-4H-(1,2,4)-triazole-3-yl-sulfanyl-N-(4-methyl-thiazole-2-yl)benzamide (CpdA);
2′,3 ′,-O-(2,4,6-Trinitrophenyl) adenosine 5′-triphosphate monolithium-trisodium salt (TNP-ATP);
2-chloroethanimidamide hydrochloride (2-Chloroacetamidine hydrochloride, 2CA). and/or
(4Z)-5-amino-6-(7-amino-6-methoxy-5,8-dioxoquinolin-2-yl)-4-(4,5-dimethoxy-6-oxocyclohexa-2,4-dien-1-ylidene)-3-methyl-1H-pyridine-2-carboxylic acid (Streptonigrin).

In one embodiment, the inhibitor or antagonist is selected from an inhibitor or antagonist of PADI4 (peptidyl arginine deiminase, type IV), such as Cl-amidine, 2-chloroacetamidine (2CA), and/or streptonigrin

In one embodiment, the inhibitor or antagonist is administered to a subject in need thereof by inhalation, intranasal, intravenous, oral, transdermal, sustained release, controlled release, delayed release, suppository, or sublingual administration.

In one embodiment, the inhibitor or antagonist is administered to a subject in need thereof in combination with a further compound, such as a further antiviral agent.

In one embodiment, more than one inhibitor or antagonist against one of PADI4, PPARδ, GCKR and/or P2X4R is administered, such as two, three, four or more inhibitors or antagonists against one of the 4 proteins.

In one embodiment, more than one inhibitor or antagonist against more than one of PADI4, PPARδ, GCKR and/or P2X4R is administered, such as
at least one inhibitor or antagonist against each one of PADI4, PPARδ, GCKR and/or P2X4R,
at least one inhibitor or antagonist against more than one of PADI4, PPARδ, GCKR and/or P2X4R, such as against two or three of the 4 proteins, and so on.

As described above, the present invention provides a pharmaceutical composition comprising
at least one inhibitor or antagonist of PADI4, PPARδ, GCKR and/or P2X4R according to the invention,
optionally, a pharmaceutical excipient,
optionally, a further antiviral agent.

In one embodiment, more than one inhibitor or antagonist against one of PADI4, PPARδ, GCKR and/or P2X4R is comprised in the pharmaceutical invention, such as two, three, four or more inhibitors or antagonists against one of the 4 proteins.

In one embodiment, more than one inhibitor or antagonist against more than one of PADI4, PPARδ, GCKR and/or P2X4R is comprised in the pharmaceutical invention, such as
at least one inhibitor or antagonist against each one of PADI4, PPARδ, GCKR and/or P2X4R,
at least one inhibitor or antagonist against more than one of PADI4, PPARδ, GCKR and/or P2X4R, such as against two or three of the 4 proteins, and so on.

As described above, the present invention provides the pharmaceutical composition the invention for use in prevention and/or treatment of infection(s) with virus(es) of the Flaviviridae family.

Preferably, the virus(es) of the Flaviviridae family are Dengue virus (DENV), Hepatitis C virus (HCV), Yellow fever virus, West Nile virus, Japanese encephalitis virus and Tick-borne encephalitis virus,

wherein the Dengue virus preferably comprises the four serotypes DENV-1, DENV-2, DENV-3 and DENV-4.

Methods for Prevention and/or Treatment of Viral Infection(s)

As described above, the present invention provides methods for the prevention and/or treatment of infection(s) with virus(es) of the Flaviviridae family.

The method comprises
administering to a patient at least one inhibitor or antagonist of PADI4, PPARδ, GCKR and/or P2X4R according to the invention or a pharmaceutical composition of the invention.

Preferably, the virus(es) of the Flaviviridae family are Dengue virus (DENV), Hepatitis C virus (HCV), Yellow fever virus, West Nile virus, Japanese encephalitis virus and Tick-borne encephalitis virus,

wherein the Dengue virus preferably comprises the four serotypes DENV-1, DENV-2, DENV-3 and DENV-4.

In one embodiment, more than one inhibitor or antagonist against one of PADI4, PPARδ, GCKR and/or P2X4R is administered, such as two, three, four or more inhibitors or antagonists against one of the 4 proteins.

In one embodiment, more than one inhibitor or antagonist against more than one of PADI4, PPARδ, GCKR and/or P2X4R is administered, such as
at least one inhibitor or antagonist against each one of PADI4, PPARδ, GCKR and/or P2X4R,
at least one inhibitor or antagonist against more than one of PADI4, PPARδ, GCKR and/or P2X4R, such as against two or three of the 4 proteins,

and so on.

In one embodiment, the inhibitor(s) or antagonist(s) or pharmaceutical composition(s) is/are administered by inhalation, intranasal, intravenous, oral, transdermal, sustained release, controlled release, delayed release, suppository, or sublingual administration.

In one embodiment, the inhibitor or antagonist is administered to a subject in need thereof in combination with a further compound, such as a further antiviral agent.

### Screening Methods

As described above, the present invention provides methods of screening for antiviral agent(s).

A screening method of the invention can comprise the following steps:

(a) adding a compound to be screened to a PADI4, PPARδ, GCKR and/or P2X4R test system, preferably a cell line expressing PADI4, PPARδ, GCKR and/or P2X4R;

(b) infecting said test system with a virus of the Flaviviridae family in the presence of said compound to be screened;

(c) removing the viral inocula and adding said compound to be screened;

(d) quantifying virus production; and

(e) comparing virus production in step (d) with the virus production in the absence of the candidate compound, wherein a difference between the measured virus productions indicates that the candidate compound is a modulator of PADI4, PPARδ, GCKR and/or P2X4R.

In particular, wherein
a decrease in the measured virus production in step (d) indicates that the candidate compound is an inhibitor or antagonist of PADI4, PPARδ, GCKR and/or P2X4R;
an increase in the measured virus production in step (d) indicates that the candidate compound is an agonist of PADI4, PPARδ, GCKR and/or P2X4R.

A screening method of the invention can comprise the following steps:

(a) infecting a PADI4, PPARδ, GCKR and/or P2X4R test system, preferably a cell line expressing PADI4, PPARδ, GCKR and/or P2X4R, with a virus of the Flaviviridae family;

(b) removing the viral inocula and adding a compound to be screened;

(c) quantifying virus production; and

(d) comparing virus production in step (c) with the virus production in the absence of the candidate compound, wherein a difference between the measured virus production indicates that the candidate compound is a modulator of PADI4, PPARδ, GCKR and/or P2X4R.

In particular, wherein
a decrease in the measured virus production in step (c) indicates that the candidate compound is an inhibitor or antagonist of PADI4, PPARδ, GCKR and/or P2X4R,
an increase in the measured virus production in step (c) indicates that the candidate compound is an agonist of PADI4, PPARδ, GCKR and/or P2X4R.

Suitable PADI4, PPARδ, GCKR and/or P2X4R test system(s) are, for example, cell(s) or cell line(s) expressing endogenously and/or ectopically PADI4, PPARδ, GCKR and/or P2X4R.

The skilled artisan is able to provide such cells or cell lines. For example, a Huh7 cell line expressing endogenously and/or ectopically PADI4, PPARδ, GCKR and/or P2X4R.

### Antiviral Targets

As described above, the present invention provides
PADI4 (peptidyl arginine deiminase, type IV),
PPARδ (peroxisome proliferator-activated receptor delta),
GCKR (glucokinase regulatory protein),

and/or
P2X4R (purinergic receptor P2X, ligand-gated ion channel 4)

for the use in diagnosing, preventing and/or treating infection(s) with virus(es) of the Flaviviridae family.

Preferably, the virus(es) of the Flaviviridae family are Dengue virus (DENV), Hepatitis C virus (HCV), Yellow fever virus, West Nile virus, Japanese encephalitis virus and Tick-borne encephalitis virus,

wherein the Dengue virus preferably comprises the four serotypes DENV-1, DENV-2, DENV-3 and DENV-4.

As described herein in further detail, the inventors have identified and validated
(1) PADI4 peptidyl arginine deiminase, type IV;
(2) PPARδ peroxisome proliferator-activated receptor delta;
(3) GCKR glucokinase regulatory protein; and
(4) P2X4R purinergic receptor P2X, ligand-gated ion channel 4.

as host cell factors (HCFs) involved in the infection of virus(es) of the Flaviviridae family, in particular DENV and HCV.

These factors are involved in the DENV replication cycle and represent key proteins implicated in the replication of other members within the Flaviviridae family.

| | nucleotide sequence | Amino acid sequence |
|---|---|---|
| PADI4 | SEQ ID NO. 1 | SEQ ID NO. 2 |
| PPARδ-4 | SEQ ID NOs. 3, | SEQ ID NOs. 4, |
| isoforms | 5, 7, 9 | 6, 8, 10 |
| GCKR | SEQ ID NO. 11 | SEQ ID NO. 12 |
| P2X4R-4 | SEQ ID NOs. 13, | SEQ ID NOs. 14, |
| isoforms | 15, 17, 19 | 16, 18, 20 |

Preferably, the invention provides PADI4, PPARδ, GCKR and/or P2X4R
as target(s) for preventing and/or treating of infection(s) with virus(es) of the Flaviviridae family,
   such as, as target(s) for antiviral treatment of DENV and similar virus infections, and/or
as screening target(s) for antiviral agent(s).

### Kits

As described above, the present invention provides a kit for diagnosing, preventing and/or treating infection(s) with virus(es) of the Flaviviridae family.

Preferably, the virus(es) of the Flaviviridae family are Dengue virus (DENV), Hepatitis C virus (HCV), Yellow fever virus, West Nile virus, Japanese encephalitis virus and Tick-borne encephalitis virus,

wherein the Dengue virus preferably comprises the four serotypes DENV-1, DENV-2, DENV-3 and DENV-4.

Said Kit Comprises
at least one inhibitor or antagonist of PADI4, PPARδ, GCKR and/or P2X4R according to the invention,
optionally, suitable substrates of PADI4, PPARδ, GCKR and/or P2X4R for in vitro enzymatic assays,
and/or
at least one of PADI4, PPARδ, GCKR and/or P2X4R.

The at least one of PADI4, PPARδ, GCKR and/or P2X4R can be provided in foiin of
nucleic acid(s), such as contained in plasmids or other expression constructs,
cells or cell lines, such as cell line(s) expressing endogenously and/or ectopically PADI4, PPARδ, GCKR and/or P2X4R,
recombinant PADI4, PPARδ, GCKR and/or P2X4R.

Preferably, the kit of the present invention is a kit for diagnosing infection(s) with virus(es) of the Flaviviridae family

In one embodiment, the kit can be used for in vitro enzymatic assays suitable to measure activity of said inhibitor or antagonist of PADI4, PPARδ, GCKR and/or P2X4R. In said embodiment, the kit can comprise protein (PADI4, PPARδ, GCKR and/or P2X4R), inhibitor(s) or antagonist(s) and the substrate(s).

Said kit can further comprise a PADI4, PPARδ, GCKR and/or P2X4R test system, such as a cell line expressing PADI4, PPARδ, GCKR and/or P2X4R, preferably expressing PADI4, PPARδ, GCKR and/or P2X4R endogenously and/or ectopically.

The kit can optionally comprise excipient(s) and further compounds.

### Preferred Embodiments

Dengue viruses (DENV) are emerging mosquito-borne human pathogens whose infections produce a variety of clinical outcomes ranging from asymptomatic to the life-treating forms dengue hemorrhagic fever/dengue shock syndrome. Recent calculations based on modeling indicate that the annual global incidence of dengue would surpass the World Health Organization estimates by more than three-fold, reaching 390 million infections per year. In spite of the efforts undertaken during the past years, there are no approved therapies to prevent or inhibit DENV infections. Due to their limited genome size, viruses exploit host factors and pathways to promote their own replication, what opens a therapeutic avenue wherein host molecules represent new targets for antiviral drugs.

With the aim to identify novel host cell determinants enhancing or restricting DENV replication, the inventors performed a high-throughput small interfering RNA (siRNA) screen in which a total of 9,102 human genes known or predicted to be potential therapeutic targets were interrogated for their effect on any step of the DENV replication cycle, i.e. entry/replication or assembly/release of progeny virus. About 100 candidate genes selected from the primary screen were re-evaluated in a secondary confirmatory screen, using a different set of siRNAs. In this way, the inventors could confirm some host cell factors and pathways, already implicated in flavivirus infection (e.g. the vacuolar ATPase, dynamin or the proteasome), but more importantly, identify novel cellular components affecting DENV replication. Among the 29 host factors validated in the secondary siRNA screen, 8 candidate proteins for which well-characterized commercially available chemical inhibitors or agonists are available were selected for further evaluation. A total of 16 compounds were analyzed for their effect on cell viability and production of infectious DENV particles.

Using this approach a total of 4 candidate host cell factors (HCFs) could be validated:
(1) PADI4 peptidyl arginine deiminase, type IV;
(2) PPARδ peroxisome proliferator-activated receptor delta;
(3) GCKR glucokinase regulatory protein; and
(4) P2X4R purinergic receptor P2X, ligand-gated ion channel 4.

### (1) PADI4

Identified target: Homo sapiens peptidyl arginine deiminase, type IV (PADI4)

GenBank Accession number (nucleotide): NM_012387 SEQ ID NO. 1

GenBank Accession number (protein): NP_036519 SEQ ID NO. 2

Peptidylarginine deiminases (PADIs) are a family of enzymes that catalyze the post-translational modification of protein arginine residues to the non-coded amino acid citrulline in a calcium- and sulfhydryl group-dependent manner (Arita et al. 2004). The PADI family in human and mouse contains five members, PADI1-4 and PADI6, that show tissue and substrate specificity. PADI2 is expressed in many tissues, including muscle, brain, and mammary gland where it citrullinates myelin basic protein and actin (Darrah et al 2012; Vossenaar et al 2003). PADI1 is expressed in the epidermis and uterus, while PADI3 and PADI6 expression seems to be restricted to hair follicles and eggs, respectively. Under physiological conditions, human PADI4 (also known as PADI5) is mainly expressed in haematopoietic cells, such as peripheral blood neutrophils, monocytes and macrophages (Wang & Wang 2013). PADI4 is the only PADI family member containing a nuclear localization signal and citrullinates many nuclear substrates including histones H3, H2A, and H4, p300/CREB-binding protein, nucleophosmin, ING4, and nuclear lamin C to exert various functions (Lee et al 2005; Wang et al 2004; Zhang et al 2011). Dysregulated PADI4 activity has been implicated in the development of several pathologies including autoimmunity, cancer, thrombosis and neurodegenerative disorders (Wang & Wang 2013; Martinod et al 2013, Bicker & Thompson 2013).

The compound N-α-benzoyl-N5-(2-chloro-1-iminoethyl)-L-ornithine amide (Cl-amidine) is a cell-permeable pan PADI inhibitor. Studies on the mode of action of Cl-amidine revealed that this compound is an inhibitor of PADI4 deimination activity with an IC₅₀ value of 5.9 μM in in vitro enzymatic assays (Luo et al 2006). This compound was also shown to inhibit PADI1 and PADI3 with IC₅₀ values of 0.8 μM and 6.2 μM, respectively (Knuckley et al 2010). The current model suggests that Cl-amidine functions by forming a covalent bond with cysteine 645 in lieu of the amidino-Cys intermediate formed during normal catalysis (Luo et al 2006). Due to the covalent nature of the bond, Cl-amidine inhibition is irreversible.

Herein it was shown that Cl-amidine is effective against DENV-2 infection in Huh7 cells (EC50: 16.09 μM) without exerting cytotoxic effect up to 1 mM. In addition, pharmacological inhibition of PADI4 proved to suppress DENV replication across all 4 serotypes and in primary human monocytes, one of the primary target cells during infection in vivo.

For the further targets, the inventors found that

(i) they play an important role in the replication cycle of DENV and

(ii) their pharmacological inhibition results in a decrease of virus production.

For details, see below as well as the Examples and Figures.

### (2) PPARδ

Identified target: Peroxisome proliferator-activated receptor delta (PPARδ)

This gene encodes for 4 different variants that result from alternate splicing:

Peroxisome proliferator-activated receptor delta isoform 1

GenBank Accession number (nucleotide): NM_006238.4 SEQ ID NO. 3

GenBank Accession number (protein): NP_006229.1 SEQ ID NO. 4

Peroxisome proliferator-activated receptor delta isoform 2

GenBank Accession number (nucleotide): NM_177435.2 SEQ ID NO. 5

GenBank Accession number (protein): NP_803184.1 SEQ ID NO. 6

Peroxisome proliferator-activated receptor delta isoform 3

GenBank Accession number (nucleotide): .NM_001171819.1 SEQ ID NO. 7

GenBank Accession number (protein): NP_001165290.1 SEQ ID NO. 8

Peroxisome proliferator-activated receptor delta isoform 4

GenBank Accession number (nucleotide): NM_001171820.1 SEQ ID NO. 9

GenBank Accession number (protein): NP_001165291.1 SEQ ID NO. 10

The following PPARδ antagonist was used:

Trivial Name: GSK0660

IUPAC name: 3-(((2-Methoxy-4-(phenylamino)phenyl)amino)sulfonyl)-2-thio-phenecarboxylic acid methyl ester

Description: Potent PPARβ/δ antagonist. GSK0660 is nearly inactive on PPARα and PPARγ with IC₅₀s greater than 10 μM.

Catalog No./Provider: G5797 (sigma)

Maximum effect on DENV replication: about 62% inhibition (see FIG. 3B)

The following PPARδ agonist was used:

Trivial Name: GW0742

IUPAC name: 4-[2-(3-Fluoro-4-trifluoromethyl-phenyl)-4-methyl-thiazol-5-yl-methylsulfanyl]-2-methyl-phenoxy}-acetic acid

Description: Highly selective PPARδ agonist. EC₅₀=1 nM vs 1 and 2 mM for PPARα and PPARγ, respectively.

Catalog No./Provider: G3295 (sigma)

Maximum effect on DENV replication: about 48% enhancement (see FIG. 3B)

### (3) GCKR

Identified target: Homo sapiens glucokinase (hexokinase 4) regulator (GCKR)

GenBank Accession number (nucleotide): NM_001486 SEQ ID NO. 11

GenBank Accession number (protein): NP_001477.2 SEQ ID NO. 12

The following GCKR inhibitor was used:

Trivial Name: Cpd A

IUPAC name: 2-Amino-5-(4-methyl-4H-(1,2,4)-triazole-3-yl-sulfanyl-N-(4-methyl-thiazole-2-yl)benzamide

Description: A cell-permeable thiazolylamide that stabilizes the glucokinase in an active conformation and prevents its interaction with and nuclear sequestration by GCKR

Catalog No./Provider: 346021 (Merck)

Maximum effect on DENV replication: about 68% inhibition (see FIG. 3B)

### (4) P2X4R

Identified target: Homo sapiens purinergic receptor P2X, ligand-gated ion channel, 4 (P2RX4) This gene encodes for 4 different variants that result from alternate splicing:

P2X purinoceptor 4 isoform 1

GenBank Accession number (nucleotide): NM_001256796.1 SEQ ID NO. 13

GenBank Accession number (protein): NP_001243725.1 SEQ ID NO. 14

P2X purinoceptor 4 isoform 2

GenBank Accession number (nucleotide): NM_002560.2 SEQ ID NO. 15

GenBank Accession number (protein): NP_002551.2 SEQ ID NO. 16

P2X purinoceptor 4 isoform 3

GenBank Accession number (nucleotide): NM_001261397.1 SEQ ID NO. 17

GenBank Accession number (protein): NP_001248326.1 SEQ ID NO. 18

P2X purinoceptor 4 isoform 4

GenBank Accession number (nucleotide): .NM_001261398.1 SEQ ID NO. 19

GenBank Accession number (protein): NP_001248327.1 SEQ ID NO. 20

The following P2X4R antagonist was used:

Trivial Name: TNP-ATP hydrate

IUPAC name: 2′,3′,-O-(2,4,6-Trinitrophenyl) adenosine 5′-triphosphate monolithium-trisodium salt

Description: Purinoceptor P2X antagonist

Catalog No./Provider: T4193 (sigma)

Maximum effect on DENV replication: about 87% inhibition (see FIG. 3B)

The following P2X4R agonist was used:

Trivial Name: Bz-ATP

IUPAC name: 2′(3′)-O-(4 Benzoylbenzoyl)adenosine 5′-triphosphate triethylammonium salt

Description: Selective P2X purinergic agonist

Catalog No./Provider: B6396 (sigma)

Maximum effect on DENV replication: about 46% enhancement (see FIG. 3B)

Discussion

The advent of RNAi-based large-scale loss-of-function screening approaches facilitated the systematic interrogation of known and predicted gene products for their effect on the replication cycle of viruses. Taking advantage of this technology, in the present invention a large-scale RNAi-based screen was used to identify host factors involved in the DENV replication cycle. The screen was based on a siRNA library targeting 9,102 known and predicted drug targets, representing approximately 40% of the entire human genome. Silencing of the expression of genes important for cell proliferation or viability can lead to dramatic alterations in cell counts and in consequence to an increased rate of false positive hit candidates. Therefore, the present invention used a Huh7-derived cell line engineered by the inventors to stably express Firefly luciferase as a surrogate marker for cell count. Quality control analysis of the primary screen revealed a high correlation between replicates (Pearson correlation coefficient >0.7), good separation of control siRNA duplexes and only minor spatial effects, which could be compensated by normalization procedures applied during statistical analysis. These data indicate a robust performance and high quality of the used screening strategy in both stages of the assay (Part I and Part II, i.e. entry/replication and assembly/release, respectively). The quality of the present study was further corroborated by the identification of cellular components previously identified in other flavivirus RNAi screens, such as vATPase and the proteasome (Krishnan et al 2008), or host factors known to be involved in the DENV life cycle such as dynamin2 and Ahrgef11 (Acosta et al 2009, Folly et al 2011).

False positives hits may also arise in siRNA screens because of nonspecific downregulation of unintended targets. In this case, the impact of such false positives can be partially overcome by the use of multiple redundant non-overlapping siRNAs. Therefore, we performed a secondary confirmatory screen of selected hits using a set of 4 different siRNAs acquired form a different supplier, which also allows excluding undesired effects caused by the particular chemical modifications of used siRNAs. Finally, in order to critically define candidate genes, we applied rather stringent hit calling criteria, requiring at least three independent siRNAs with a z-score ≦−2 or ≧2 and a p-value ≦0.05. Using these thresholds, we aimed at the identification of “high-confidence” candidates, minimizing the occurrence of false positive hits.

Taking advantage of the fact that the used siRNA library was designed to silence known or predicted therapeutic targets, we performed an orthogonal screen using chemical compounds that modulate (inhibit or activate) the function of the identified proteins for the validation of our hits. The effect of each drug on cell viability was first determined using two different approaches. Only compounds and concentrations reducing cell viability less than 20% (as determined by both methods) were used for the subsequent analysis in the infection assays. In this way we ensure that the observed effect on virus production is not due to an indirect effect of the compounds on cell growth or the metabolic state. In this way, 4 proteins (GCKR, PPARδ, PADI4 and P2X4R) were found to be required for the DENV life cycle and were suitable as drug targets as demonstrated by reduced virus production upon pharmacological inhibition of each of these proteins. Interestingly, re-evaluation of the effect of these compounds against the related HCV that also belongs to the family Flaviviridae and the distantly related Vesicular Stomatitis Virus (VSV) belonging to the family Rhabdoviridae, revealed a requirement of the identified proteins for HCV, but not VSV replication, arguing for the potential of these drugs as pan-flaviviral inhibitors. In fact, in case of PPAR, pharmacological inhibition of another member of the PPAR family, PPARα was shown to reduce HCV RNA replication and production of infectious particles (Gastaminza et al 2010) and the level of P2X4R transcripts was found to be increased in Huh7 cells stably expressing HCV envelope proteins (Manzoor et al 2011).

To confirm that the inhibitory effect exerted by Cl-amidine was due to its direct effect on PADI4 rather than on an alternative enzyme which may share similar activity, we generated a stable cell line overexpressing PADI4 and analyzed the effect of the drug on virus replication by repeating the virus yield inhibition assay. We observed an about 10-fold difference in the EC₅₀ value upon PADI4 overexpression, confirming Cl-amidine indeed targets this protein and that inhibition of this protein is the cause for reduced DENV replication/virus production. The potential of PADI4 as a novel antiviral target is highlighted by our observations that Cl-amidine exerts inhibitory action against DENV-2 in primary human monocyes, one of the primary target cells during infection in vivo. Moreover, inhibition of PADI4 blocks replication of DENV independent of the serotype. This is of utmost important because of the rapid geographic expansion and increasing co-circulation of the four serotypes. Finally, inhibition of PADI4 also suppresses replication of HCV, thus demonstrating the suitability of PADI4 as a target for broad-spectrum antiviral drugs blocking members of the Flaviviridae.

### Further Preferred Embodiments

The inventors provide furthermore new evidence that further corroborates that PADI4 is a host dependency factor for DENV replication. We found that reducing the expression levels of PADI4 in cell cultures reduces DENV replication and enhances the inhibitory effect of Cl-amidine, a PADI4 inhibitor. In addition, we demonstrate that PADI4 is required at an early step of DENV replication that leads to genome amplification and that the intracellular distribution of this host protein is modified by DENV from nucleolar to a difuse cytoplasmic distribution. Finally, we provide evidence that additional compounds targeting PADI4 activity are able to reduce DENV production, increasing the list of molecules that can be included in a hit-to-lead optimization process that could lead to the discovery of potent anti-dengue drugs suitable for antiviral therapy.

Discussion

As discussed above, peptidylarginine deiminases (PADI) are a family of enzymes that catalyze the post-translational modification of protein arginine residues to the non-coded amino-acid citrulline in a calcium- and sulfhydryl group-dependent manner. The PADI family in human and mouse contains five members, PADI1-4 and PADI6, that show tissue and substrate specificity. Under physiological conditions, human PADI4 (also known as PADI5) is mainly expressed in hematopoietic cells, such as peripheral blood neutrophils and monocytes (Wang & Wang, 2013, Vossenaar et al 2004), the latest being the main targets of DENV infection in humans. These enzymes have a proven role in cellular differentiation; embryonic development and regulation of gene transcription (Christophorou et al 2004; Nakashima et al 2013; Kolodziej et al 2014).

However in recent years PADI4 and PADI2 have drawn interest given their involvement in several autoimmune diseases, such as rheumatoid arthritis, inflammatory bowel disease, Alzheimer's disease, multiple sclerosis, lupus erythematodes, Parkinson's disease and cancer.

Given the observed link between PADI4 overexpression and upregulated enzyme activity and the development of several pathological states, the in vivo efficacy of Cl-amidine for the treatment of such syndromes have been evaluated. Cl-amidine administration was shown to be immunomodulatory and to successfully reduce disease severity in mouse models of lupus (Knight et al 2013), rheumatoid arthritis (Willis et al 2011), colitis (Chumanevich et al 2011) and arthrosclerosis (Knight et al 2014) and to reduce tumor growth in a breast cancer model (McElwee et al 2011), among others.

As discussed above and herein, we herein report the identification of PADI4 as a host factor involved in DENV replication. In addition, we highlight the potential of this cellular protein as a novel target for antiviral design by demonstrating that Cl-amidine exerts inhibitory action against DENV-2 in primary human monocyes, one of the primary target cells during infection in nature, and throughout the four DENV serotypes. The further results included herein (see e.g Example 2) reinforce the notion that PADI4 is a host dependency factor required for DENV replication and demonstrate that targeting the enzymatic activity of PADI4 using Cl-amidine is specific and has a negative impact on virus genome replication at early stages of viral life cycle:
1. Silencing of PADI4 reduces replication and production of DENV and the combined used of Cl-amidine and shRNA-mediated gene know-down of PADI4 have a synergistic effect of DENV production (FIG. 7).
2. Time-of-addition experiments using DENV particles that are able to undergo one single round of infection indicate that Cl-amidine induces a strongest inhibitory effect when added before infection and at early time points after infection (FIG. 8 A).
3. Bypassing the step of virus entry by directly delivering viral RNA molecules into the cell cytoplasm by transfection demonstrates that Cl-amidine targets a post-entry step in the DENV life cycle (FIG. 8 B).
4. Evaluation of the effect of Cl-amidine on translation and replication of incoming viral RNA molecules demonstrates that PADI4 is required for replication of genomic RNA molecules but not for translation of viral proteins (FIG. 8 C).
5. Direct comparison of the effect of Cl-amidine in newly transfected cells with cells in which replication has reached steady-state indicate that PADI4 is required at the onset of viral genome replication (FIG. 8 D).
6. PADI4 is redistributed to the cytoplasm in response to DENV infection (FIG. 9).
7. In addition to Cl-amidine, 2CA and streptonigrin molecules that inhibit PADI4 activity, exert antiviral effect against DENV. Both compounds displayed activity at nontoxic concentrations and therefore have therapeutic potential (FIG. 10).

Taken together, these results illustrate the ability of our screening system to identify inhibitors that target known and unknown aspects of DENV life cycle. Our findings not only provide a set of chemical tools to study events in virus replication that might otherwise remain elusive, but also provide new avenues for the development of novel therapeutic agents against DENV and other member of the family Flaviviridae.

The following examples and drawings illustrate the present invention without, however, limiting the same thereto.

### BRIEF DESCRIPTION OF THE DRAWINGS

FIG. 1. Identification of novel host cell factors involved in DENV infection using high throughput siRNA screens and an orthogonal validation screen.

Outline of the validation process carried out to discover novel host factors involved in DENV infection.

FIG. 2. Results from primary and secondary siRNAi screens.

A. siRNA z-score in primary screen.

Individual siRNAs are ranked according to their mean z-scores over all three replicates from lowest (host dependency factors: HDFs) to highest (host restriction factors: HRFs). Known DENV-host factors, as well as several selected newly identified genes are indicated for both parts of the screen. For each gene only the top-scoring siRNA is highlighted, irrespective of the total number of scoring siRNAs for a given gene.

B. z-scores of hits validated after secondary screen. Error bars represent the standard deviation of the mean z-scores of different siRNAs.

FIG. 3. Orthogonal validation screen using chemical inhibitors/activators.

A. Assay set up of chemical compounds screen.

Pretreatment conditions: Huh7 cells were pretreated for 2 h with each compound and infected with DENV-2 (strain 16681) at a multiplicity of infection (MOI) of 0.1 PFU/cell in the continued presence of the drugs. After 1 h of infection, viral inocula were replaced with fresh culture medium containing compounds.

Post-treatment conditions: Huh7 cells were infected with DENV-2 at a MOI of 0.1 PFU/cell in the absence of the compounds. After 1 h of infection, viral inocula were replaced with fresh culture medium containing the compounds. In both assay conditions virus titers were determined 48 h after infection by plaque formation assay.

B. Impact of small molecule inhibitors on DENV production. Results represent the mean±SD of three independent experiments.

C. Similar conditions as in (B) were used to analyze the effect of selected drugs on HCV (JC1) and VSV infection in Huh7.5 or Huh7 cells, respectively. HCV virus production was quantified by limiting dilution assay (TCID₅₀), while VSV production was measured by plaque assay.

FIG. 4. The cell-permeable compound Cl-amidine is specifically targeting PADI4, and inhibits DENV infection.

A. Cl-amidine treatment reduces production of infectious DENV particles in Huh7 cells. Parental Huh7 cells or the stable cell lines Huh7-Empty and Huh7-PADI4 were treated for 2 h with increasing concentrations of Cl-amidine and then infected with DENV-2 at a MOI of 0.1 PFU/cell. After 1 h infection in the presence of the compound viral inocula were removed and cells dfurther cultivated in the presence of the drug. Viral titers were determined after 48 h of infection by plaque formation assay. Median effective concentrations (EC₅₀) and fold shift values in PADI4 overexpressing cells are given in Table 2. *p<0.05; **p<0.01; ***p<0.001. Grey stars indicate significant inhibition with respect to DMSO-treated cultures, while black stars denote a significantly different inhibitory effect in Huh7-PADI4 with respect to Huh7-empty cultures.

B. Cl-amidine does not exert cytotoxic effect in Huh7 cells. Huh7 cells were treated with increasing concentrations of Cl-amidine and after 48 h cell viability was determined by quantification of ATP content in cells.

C. Overexpression of PADI4 in stable cell lines. Cell pools of Huh7 cells stably overexpressing PADI4 (Huh7-PADI4) or a control cell line (Huh7-Empty) were generated by lentiviral transduction. The overexpression of the protein was confirmed by indirect immunofluorescence assay using an anti-PADI4 specific antibody.

FIG. 5. PADI4 as a potential target for antiviral therapy.

A. Cl-amidine exerts antiviral activity against DENV-2 in primary human monocytes. Primary human monocytes isolated from peripheral blood from healthy donors were infected with DENV-2 at an MOI of 10 PFU/cell in the presence of the drug. Viral inocula were removed 3 h after infection and fresh medium containing Cl-amidine was added. Viral titers were determined 48 h after infection by plaque assay. Calculated EC₅₀ values for each donor are summarized in Table 3.

B. Cl-amidine has no negative impact on viability of primary human monocytes. Viability of purified monocytes was determined 48 h after Cl-amidine treatment by measuring ATP content.

C. Cl-amidine shows a similar inhibitory effect against the four DENV serotypes. The effect of Cl-amidine (200 μM) against the four DENV serotypes in Huh7 cells was assessed by a virus yield inhibition assay 48 h after infection.

FIG. 6. Viability of Huh7 cells after exposure to different chemical compounds in orthogonal validation screen.

Huh7 or Huh7-F-Luc cells grown in 96-well plates were treated with increasing concentrations of each compound and viability was determined after 48 h by measuring ATP content (in Huh7 cells) or F-Luc activity (in Huh7-FLuc cells). Note that concentrations chosen to carry out the validation experiments correspond to those in which cell viability was higher than 80% in both methods used for cell viability.

FIG. 7. Reducing the expression levels of PADI4 enhances the susceptibility of DENV to Cl-amidine.

Huh7 cells transduced with lentiviral vectors expressing irrelevant sequences (shNT) or shRNAs targeting PADI4 mRNA were infected with DV-R2A. 48 h after infection virus replication was determined by measuring RLuc activity from cell lysates (A), while virus production was quantified from cell supernatants by plaque assay (B). The impact of PADI4 silencing on cell viability was determined 48 and 96 h post-transduction by quantifying ATP content using a bioluminescent assay (C). Huh7 cells transduced with lentiviruses expressing an irrelevant shRNA (shNT#2) or shPADI4#4 were infected with DENV-2 in the presence or absence of increasing concentrations of Cl-amidine. Virus production was quantified 48 h after infection by plaque assay. The dose-response curve obtained in parental Huh7 cells is represented as a black dashed line (D). The effect of Cl-amidine treatment on transduced cells was determined 48 h after treatment by measuring intracellular ATP content (E).

FIG. 8. Cl-amidine inhibitory effect is exerted at the onset of DENV replication.

A. Huh7 cells were infected with DENV_{TCP} carrying a subgenomic Renilla-reporter. After 1 h of incubation at 37° C. the inoculum was removed and fresh medium was added. At time points specified at the bottom of the graph, Cl-amidine was added at a final concentration of 200 μM. In one case (2 h), cells were pretreated with Cl-amidine for 2 h prior to infection. Cells were harvested 48 h postinfection, and Rluc activities in the lysates were measured.

B. In vitro transcribed RNA of a DENVsg reporter replicon were introduced into Huh7 cells by electroporation and Cl-amidine (200 μM) was added immediately to the cells. Rluc activity was determined from cell lysates 48 h after transfection. *p<0.05; **p<0.01; ***p<0.001.

C. Huh7 were transfected with in vitro transcribed RNAs of the DENVsg reporter replicon or a mutant carrying a mutation in the polymerase (DENV(GND)sg). Cl-amidine (200 μM) was added immediately after transfection. At different time points cells were harvested and Rluc activity was determined.

D. Huh7 cells were electroporated with a DENVsg reporter replicon (newly stablished) while Huh7 cells carrying a stable DENVsg replicon (stady-state) were mock electroporated. Cl-amidine (200 μM) or DMSO were added immediately to the cells and Riuc activity was determined at different time points after transfection.

FIG. 9. Endogenous PADI4 is redistributed upon DENV infection.

A. Huh7 cells were infected with DENV-2 at a multiplicity of infection (moi) of 1 PFU/cell. 48 h after infection cultures were fixed and stained using a PADI4 immune serum followed by AlexaFluor488 IgGs

B. Quantification of signal intensities in nucleus and cytoplasm was performed in 50 cells.

FIG. 10. 2CA and Streptonigrin inhibit DENV production at non-cytotoxic concentrations.

A. Huh7 cells were treated with increasing concentrations of 2CA or streptonigrin. Cell viability was determined 48 h after treatment by quantification of intracellular ATP content.

B. Huh7 cells were pretreated or not with streptonigrin (12 nM) or 2CA (50 μM) for 2 h and then were infected in the presence or absence of the inhibitors. Viral inocula were removed 1 h after infection and cultures were covered with fresh medium containing the drugs. Viral titers were determined 48 h after infection by plaque assay. Results are expressed as percentage with respect to DMSO treated cultures ±SD. *p<0.05; **p<0.01; ***p<0.001.

### EXAMPLES

### Example 1

### 1. Materials and Methods

### 1.1 Cells and Viruses

The human hepatocarcinoma cell line Huh7, African green monkey kidney cells (Vero E6), Baby hamster kidney cells (BHK-21), human embryonic kidney cells (HEK293T) and the stable cell lines Huh7-Fluc and Huh7-PADI4 were cultivated in Dulbecco's modified minimal essential medium (DMEM; Life Technologies, Frankfurt, Germany) supplemented with 2 mM L-glutamine, non-essential amino acids, 100 U/ml penicillin, 100 μg/ml streptomycin, and 10% fetal calf serum (FCS) (DMEMcplt). Huh7-Fluc and Huh7-PADI were derived from Huh7 cells by lentiviral transduction of genes encoding for Firefly luciferase or human PADI4 (see below).

Cell Lines Used in this Study:

| Cell line | Organism and tissue |
|---|---|
| Huh7 | Human hepatocarcinoma |
| VeroE6 | Cercopithecus aethiops kidney |
| BHK-21 | Baby hamster kidney |
| HEK293T | Human embryonic kidney. Constitutively expresses the |
| | simian virus 40 (SV40) large T antigen |

The Renilla luciferase reporter DENV-2 16681 (DV-R2A) which encodes a Renilla luciferase that is fused N-terminally to the 32 N-terminal residues of the capsid protein and C-terminally to the 2A peptide of Thosea asigna virus was described before (Fischl & Bartenschlager 2013). Virus stocks (DV-R2A or DENV wild type) were generated by transfection of in vitro transcribed viral RNA into BHK-21 cells by electroporation (seed stocks) followed by one round of amplification in Vero cells (Fischl & Bartenschlager 2013).

### 1.2 Compounds and Reagents

Alda-1 (126920), Cpd A (346021) and Cl-amidine (506282) were obtained from Merck. Daidzin (30408), Atropine (A0257), Methoctramine hydrate (M105), Oxotremorine E (O100), Methylglutamic acid (G137), Kainic acid monohydreate (K2389), TNP-ATP hydrate (T4193), Bz-ATP (B6396), GSK0660 (G5797), GW0742 (G3295), Capsazepine (C191), Menthol (M2772) and WS-12 (W0519) were purchased from Sigma-Aldrich.

### 1.3 High-Throughput RNAi Screens.

Preparation of 384-plates for solid-phase reverse transfection was carried out in the ViroQuant-CellNetworks RNAi Screening Facility, Heidelberg. Briefly, 3 μl OptiMEM (supplemented with 0.4 M sucrose), 3.5 μl Lipofectamine™ 2000 and 5 μl of the respective siRNA stock solution (30 μM, Ambion Silencer® Extended druggable genome library V3) were mixed using an automated liquid handler (Microlab STAR, Hamilton) in 384-well low-volume plates (Nalge Nunc International) and incubated for 30 min at RT. Afterwards, 7.25 μl of a 0.2% (w/v) gelatine solution containing 0.01% (v/v) fibronectin was added and 18 μl of the resulting mix was finally diluted in 180 μl H₂Odd. Subsequently, 5 μl of the transfection mix was spotted into each well of a white μclear 384-well plate (Greiner Bio-One, Frickenhausen). Finally, plates were shortly spun down, incubated for 1 hour at 50° C. and centrifuged for 36 min at 50° C. in a centrifugal evaporator (Genevac Mivac Quattro, Thermo-Fisher). Dried multi-well plates were stored in sealed plastic boxes (EMSA, Emsdetten) containing drying pearls (Fluka, Steinheim).

Huh7-Fluc cells were seeded in the spotted 384-well plates at a density of 1.2×10³ cells per well in a total volume of 50 μl DMEMcplt. The plates were spun down twice briefly to ensure even distribution of the cells and incubated at 37° C., 5% CO₂. After 72 hours of siRNA-mediated gene knockdown, the medium was aspirated using a semi-automated liquid handler (Hydra DT, Thermo-Fisher) and cells were infected with DV-R2A at an MOI of approx. 5 PFU/cell in a total volume of 10 μl DMEMcplt per well. Four hours later, 40 μl DMEMcplt was added to each well, and the plates were allowed to incubate for further 48 hours at 37° C., 5% CO₂. Next, 50 μl of the cell culture supernatant was replica plated onto 384-well plates containing naïve Vero cells (beginning of part II), seeded 24 hours earlier at a density of 2.5×10³ cells per well (in 20 μl DMEMcplt). Huh7-FLuc cells were processed for dual-luciferase assay “Part I” while Vero cells were processed for R-Luc assay 36 hours later (Part II). Negative control siRNAs directed against GFP and the HCV genome (siHCV-321), as well as positive control siRNAs targeting different regions of the DENV genome (siDV-NS1 and siDV-NS3) were present on each plate in duplicates. In addition, wells containing either buffer alone or a scrambled control siRNA (Ambion) were spotted on all plates. The screen was performed in triplicate.

For the Secondary screen the screening procedure was adapted to a 96-well plate format with the following modifications: Huh7-Fluc cells were seeded in the spotted 96-well plates at a density of 3×10³ cells per well in a total volume of 100 μl DMEMcplt. Cells were infected with DV-R2A at an MOI of approx. 1 PFU/cell in a total volume of 20 μl DMEMcplt. After a four-hour incubation at 37° C., 5% CO₂, 90 μl DMEMcplt was added to each well. Naïve Vero cells were seeded at a density of 4×10³ cells per well in a total volume of 50 μl DMEMcplt. The next day, 70 μl cell culture supernatant from each well of part I of the screen was transferred to a new well containing the previously seeded Vero cells (Part II). The Secondary screen was performed in quadruplicates, each plate containing three negative control siRNAs directed against GFP, three predesigned non-targeting control siRNAs (ON-TARGETplus non-targeting siRNA #1, Dharmacon) and three positive control siRNAs directed against the virus genome (siDV-NS1), as well as empty wells containing buffer alone.

Statistical analysis of the data was carried out in R Version 2.8.0 (R Development Core Team, http://www.R-project.org), using the Bioconductor packages RNAither (Rieber et al 2009) and cellHTS (Boutros et al 2006). In brief, signal intensities were log-transformed and the lowest 5% based on the cell count (only for part I of the screen) were excluded from the further analysis. The signal is subsequently normalized based on the cell count using locally weighted scatterplot smoothing (only for part I). Spatial effects were removed by B score normalization (Brideau et al 2003) and z-scores were computed by subtracting the plate median from each measurement and dividing by the plate median absolute deviation. Replicates were summarized using the mean and p-value was computed using t-test.

### 1.4 Dual-Luciferase Reporter Assays

For dual luciferase measurement of Firefly- and Renilla-Luciferase in 384-well or 96-well microplates, a modified luciferase assay protocol was established, allowing processing of large batches of plates. To this end, cells were washed once with sterile PBS and lysed directly in the plate by the addition of 20 μl (384-well) or 30 μl (96-well) luciferase lysis buffer per well and freezing at −80° C. Shortly before measurement, lysates were allowed to thaw at RT for 30 to 60 min. Subsequently, Firefly assay buffer, supplemented with 70 μM D-luciferin, was added to each well using a Multidrop 384 dispenser (Thermo-Fisher), and the plates were incubated for 5 min at RT in the dark. FLuc activity was measured for 0.1 sec in a Mithras LB940 multimode microplate reader (Berthold Technologies, Bad Wildbad). After addition of the Renilla assay buffer, supplemented with 7.14 μM coelenterazine, RLuc activity was measured for 0.5 sec using a 475 nm filter in the same multiwell reader.

### 1.5 Cell Viability and Virus Yield Reduction Assays

The suitable concentration of each chemical compound to be tested in virus yield inhibition assays was first established by determining the effect of each drug on cell viability using two different methods: (1) quantification of intracellular ATP content, (2) expression of reporter FLuc gene. Briefly, 1×10⁴ Huh7 or Huh7-Fluc cells seeded in 96-well plates were treated with increasing concentrations of the compounds or vehicle control and incubated at 37° C. for 48 h. In the case of Huh7 cells viability was determined by measuring ATP content using the CellTiter-Glo® Luminescent Cell Viability Assay kit (Promega) following the manufacturer's instructions. For measurement of Firefly luciferase activity, Huh7-FLuc cells were lysed in 30 μl of luciferase lysis buffer and frozen at −80° C. After thawing lysates at RT, FLuc activity was measured for 1 sec in a Mithras LB940 multimode microplate reader by injecting 100 μl/well of luciferase assay buffer supplemented with D-Luciferin. Viability was determined as the ratio between signals in treated cultures vs vehicle control cells. Concentrations resulting in cell viability greater than 80% in both methods were chosen for subsequent virus yield inhibition assays.

The effect of each compound on virus production was assessed by a virus yield inhibition assay using DENV-2 wild type virus. Huh7 cells seeded in 24-well plates were either pretreated or not for 2 h with the drugs and then infected with DENV-2 (in presence or absence of the compounds) at an MOI of 0.1 PFU/cell. After 1 h incubation at 37° C. viral inocula were removed and replaced by DMEMcplt containing the drugs. Virus titers were determined after 48 h by plaque assay in VeroE6 cells using an overlay medium containing 1.5% carboxymethylcellulose. When applicable, EC₅₀ values were estimated by graphic interpolation of the compound concentration resulting in 50% inhibition of DENV infection in the linear portion of the curve in a % of inhibition vs log₁₀ transformed concentration of drug graph.

### 1.6 Generation of Stable Cell Lines by Lentiviral Transduction

Recombinant lentiviruses encoding FLuc or PADI4 genes were generated by co-transfection of a lentiviral expression vector plasmid along with two helper plasmids into HEK293T cells using the CalPhos™ Mammalian Transfection kit. Briefly, 1.2×10⁶ HEK293T cells plated onto 6 cm dishes were transfected with 6.4 μg lentiviral vector (pWPI-Neo-Fluc or pWPI-puro-PADI4), 6.4 μg gag-pol plasmid (pCMV-dR8.91) and 2.1 μg VSV-G envelope expression plasmid (pMD.G). After 6-16 hours, the medium was carefully replaced with 5 ml of fresh DMEMcplt and 24 h later supernatants containing infectious lentiviral particles, were harvested, filtrated using a 0.45 μM syringe tip filter and directly used to infect Huh7 cells. Transduction of target cells was performed for a total of three times to achieve a high number of integrates and thus high expression levels. Transduced cells were subjected to selection and maintained in growth medium containing 1 mg/ml G418 (for Huh7-FLuc) or 1 μg/ml puromycin (for Huh7-PADI4). Overexpression of PADI4 was corroborated by indirect immunofluorescence staining using a rabbit polyclonal antiserum directed against PADI4 (Abeam) followed by incubation with a secondary antibody coupled to Alexa-Fluor488. Cells were mounted on microscope glass slides using Fluoromount G and images were acquired using a Nikon microscope.

1.7 Purification of Primary Human Monocytes from Peripheral Blood

Peripheral blood samples from three healthy donors were collected in EDTA tubes. The blood was then diluted with 2 volumes of DPBS without calcium and without magnesium (PAN Biotech) and carefully layered onto Ficoll-hypaque (PAN Biotech). The blood was then subjected to centrifugation at 700 g, without brakes. The interphase cells containing the peripheral blood mononuclear cells (PBMCs) were aspirated, transferred to a clean tube and washed three times with DPBS. CD14+ monocytes were isolated using MicroBeads conjugated to monoclonal anti-human CD14 antibodies (MACS Miltenyi Biotec) according to the manufacturer's instructions, with some modifications. Briefly, 10 μl of Microbeads were incubated with 1×10⁷ cells for 15 min at 4° C. After washing with DPBS magnetic separation of CD14+ monocytes was performed using an autoMACS Separator. The purity of the isolated monocytes (89.8% for donor 1, 94.8% for donor 2 and 97.7% for donor 3) was corroborated by FACS staining using anti-CD14 antibodies.

### 2. Results

### 2.1 Identification of Novel Host Cell Factors Involved in DENV Replication Using RNAi Screens

In order to identify novel host proteins involved in any step of DENV infection, we performed two sequential two-step high-throughput siRNA screens (designated as “Primary screen” and “Secondary screen”) using a reporter virus system (DV-R2A) engineered to express Renilla luciferase (RLuc) in infected cells (described in detail in Fischl & Bartenschlager 2013). Conditions were optimized to detect multiple cycles of infection, resulting in 40-50% of infected cells at the end of the assay, which is expected to allow the detection of both host dependency factors (HDF) and host restriction factors (HRF).

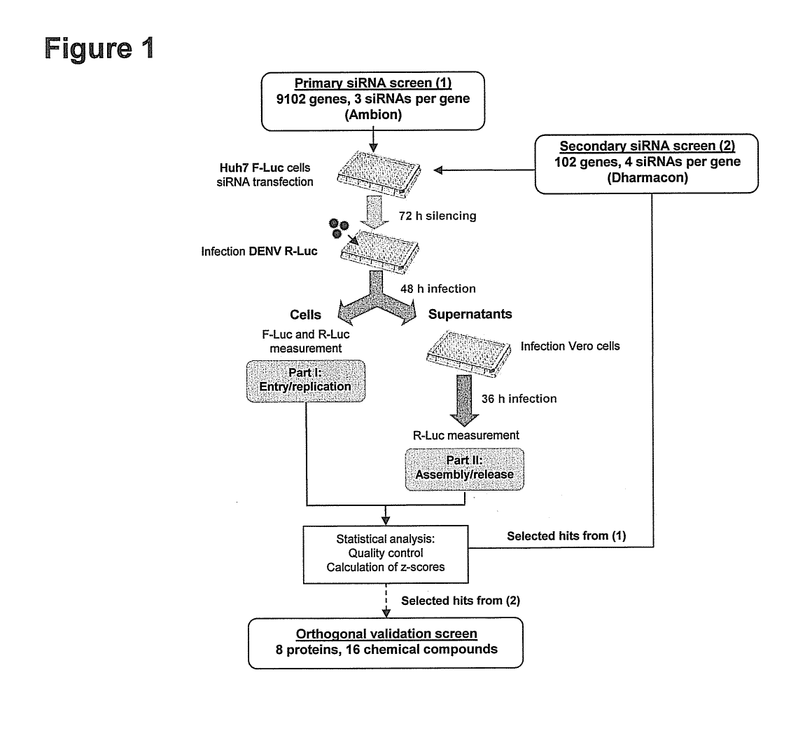

For the Primary screen, a total of 9,102 human genes known or predicted to be potential therapeutic targets where silenced in the hepatoma cell line Huh7, stably expressing Firefly luciferase (Huh7-FLuc) using 3 independent siRNAs per gene. SiRNA were solid-phase reverse transfected into Huh7-FLuc cells in 384-well plates and subsequently infected with DV-R2A (FIG. 1). After 48 h of infection F-Luc (used as surrogate marker for cell number and viability) and R-Luc (used as marker for DENV replication) were measured using a home-made dual-luciferase assay (Fischl & Bartenschlager 2013). This step of the screen is designated “Part I: Entry/replication” (FIG. 1).

To identify host factors affecting late steps in the DENV life cycle (virus production, i.e. assembly, and virus particle release), we used supernatants collected at the end of the experiment to infect naïve Vero cells and subsequently quantified RLuc activity (“Part II: Assembly/release”; FIG. 1). The complete set of siRNAs—including four positive controls targeting the DENV genome (DV-NS1 and DVNS3 in duplicate) and a negative control (siGFP) per plate—were assayed in triplicates. Finally, z-scores for each siRNA were calculated and p-values were computed using t-test. To account for cytotoxic effects, the lowest 5% of wells based on FLuc signal were excluded from the analysis (only Part I). In the present study, thresholds classifying a silenced gene as a hit candidate were set to a deviation of the virus-specific signal by at least 2 standard deviations (sd) from the plate mean for at least two different siRNAs per gene and a p-value of ≦0.05 for part I of the screen. For part II all siRNAs with a z-score in part I lower than −2 or higher than 2, respectively were eliminated and again, the same thresholds as for part I were applied. These criteria were met by 98 unique genes, corresponding to approximately 1% of the total number of genes targeted by the library.

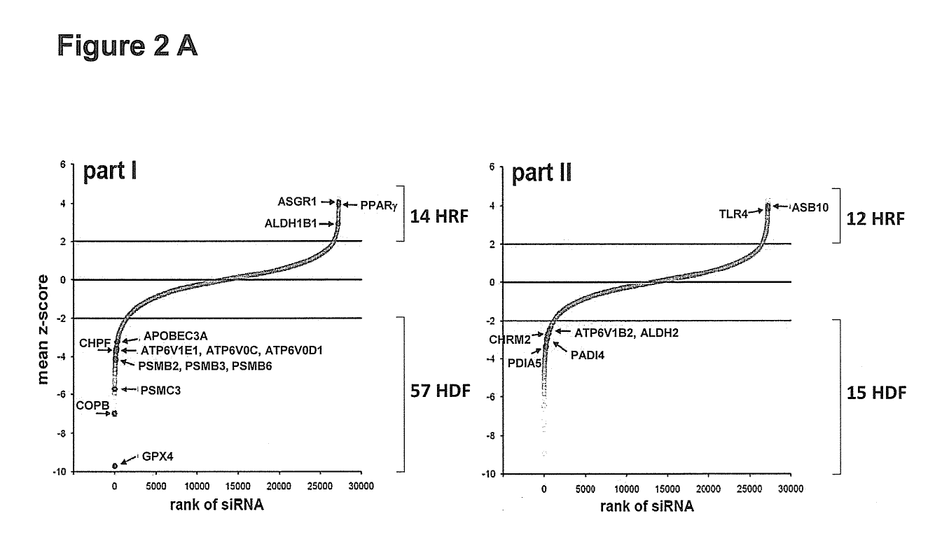

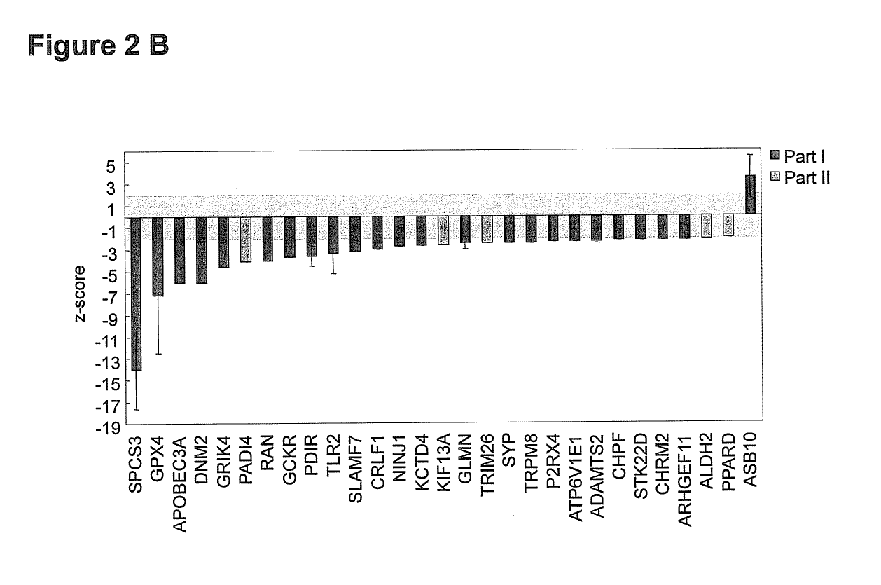

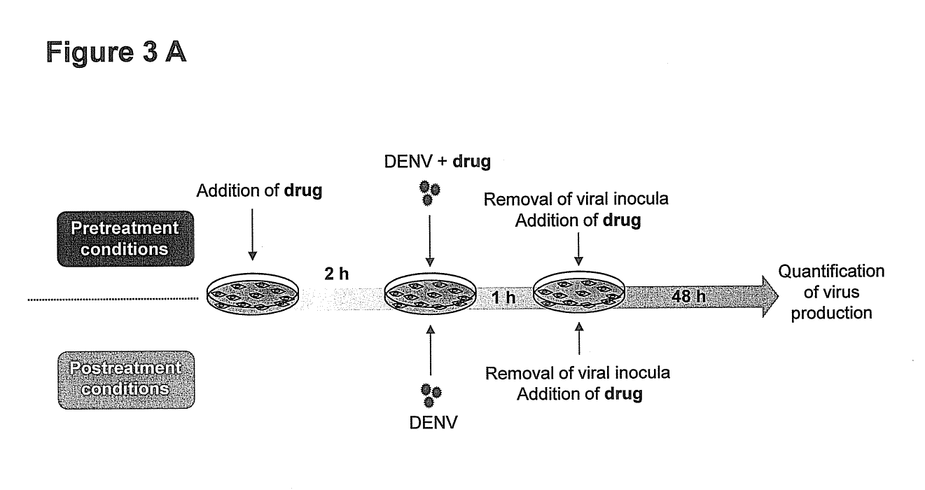

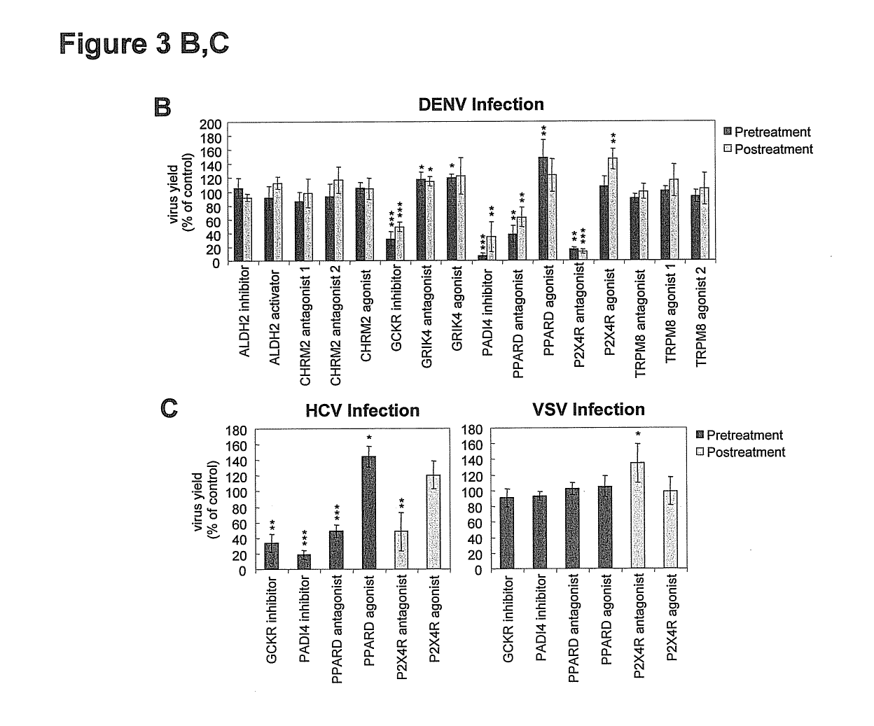

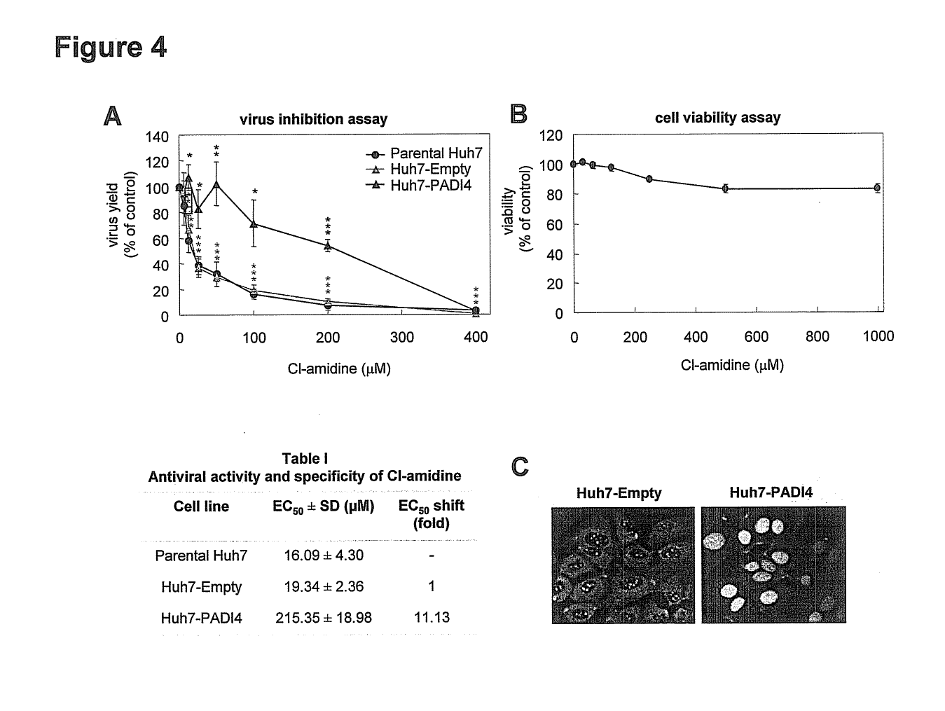

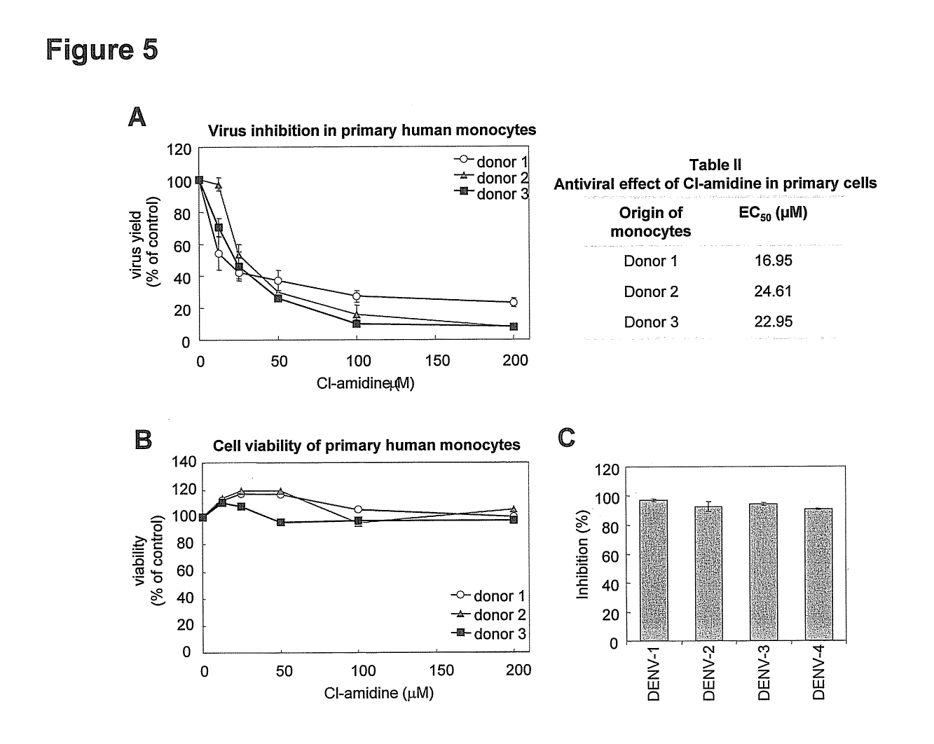

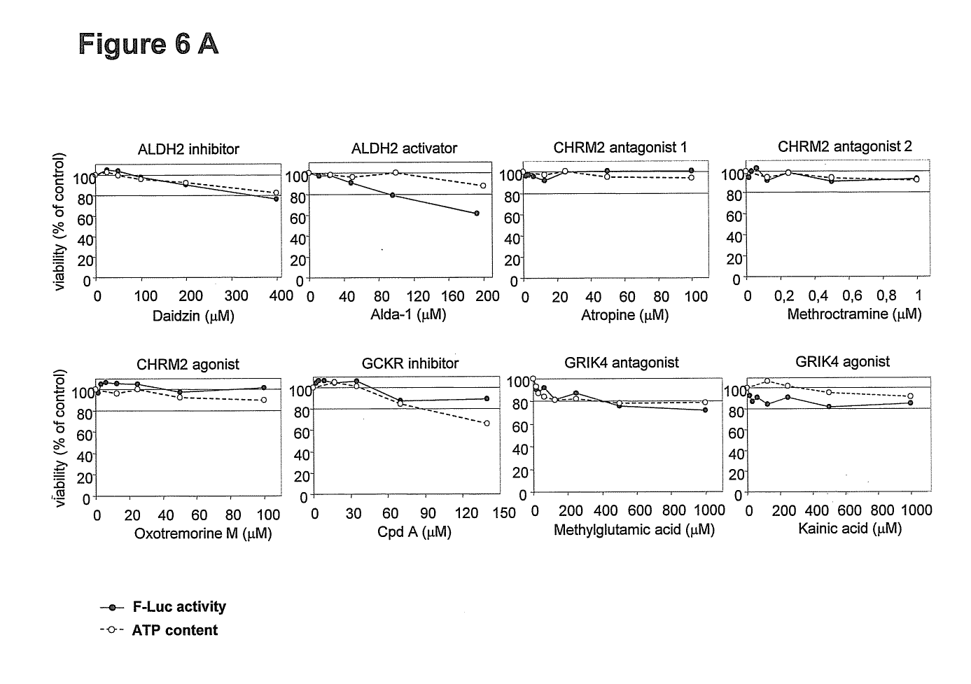

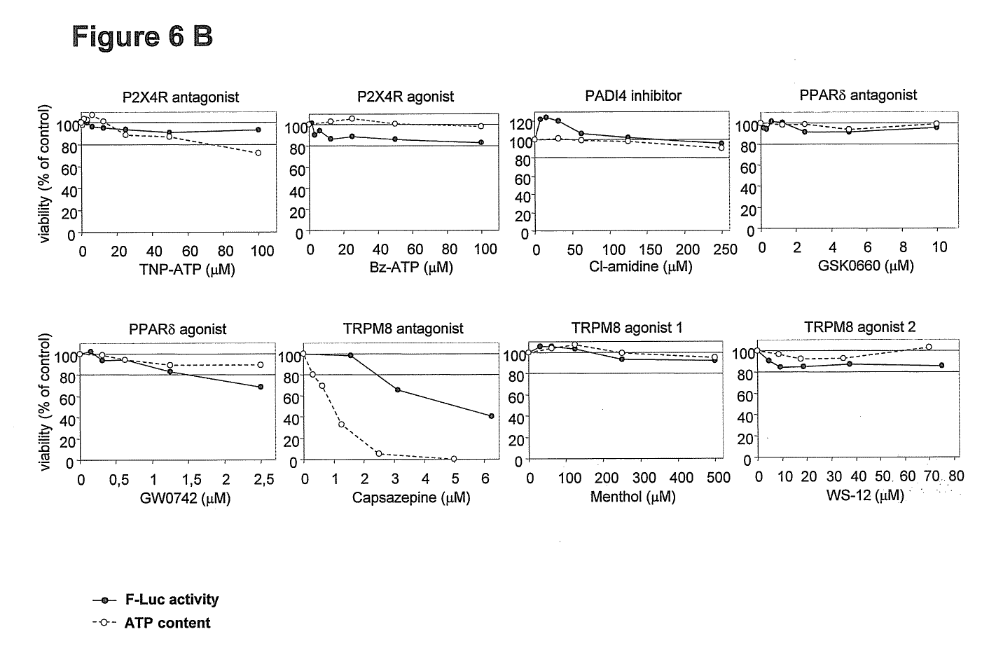

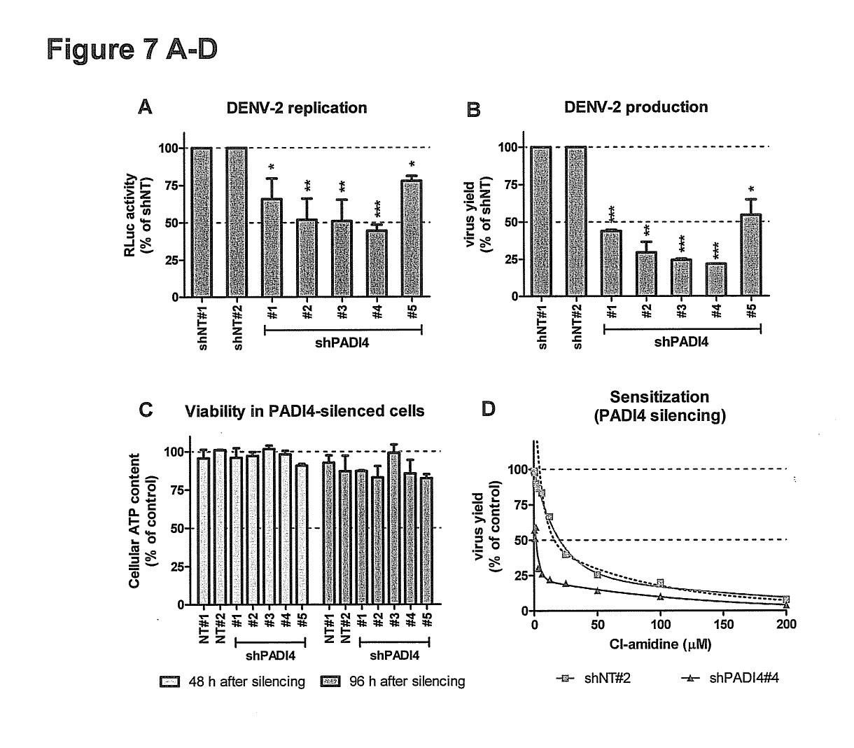

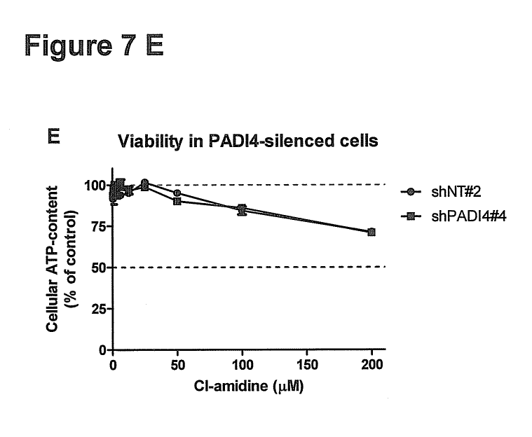

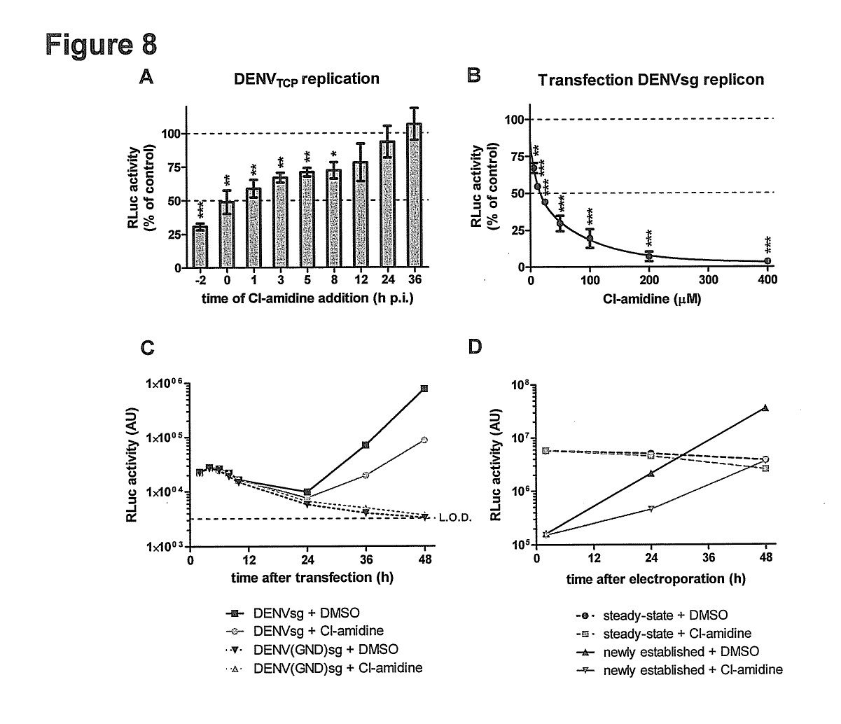

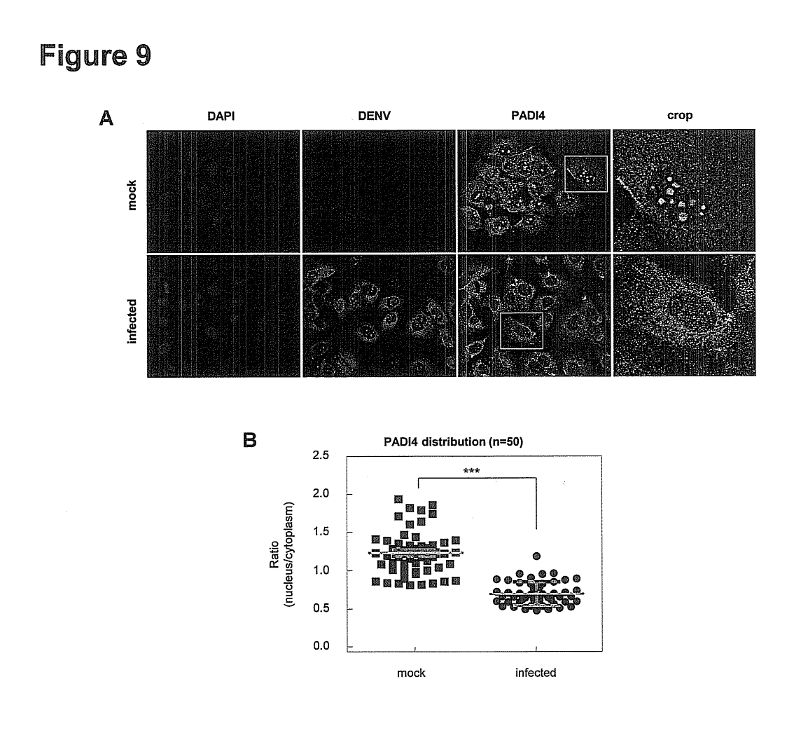

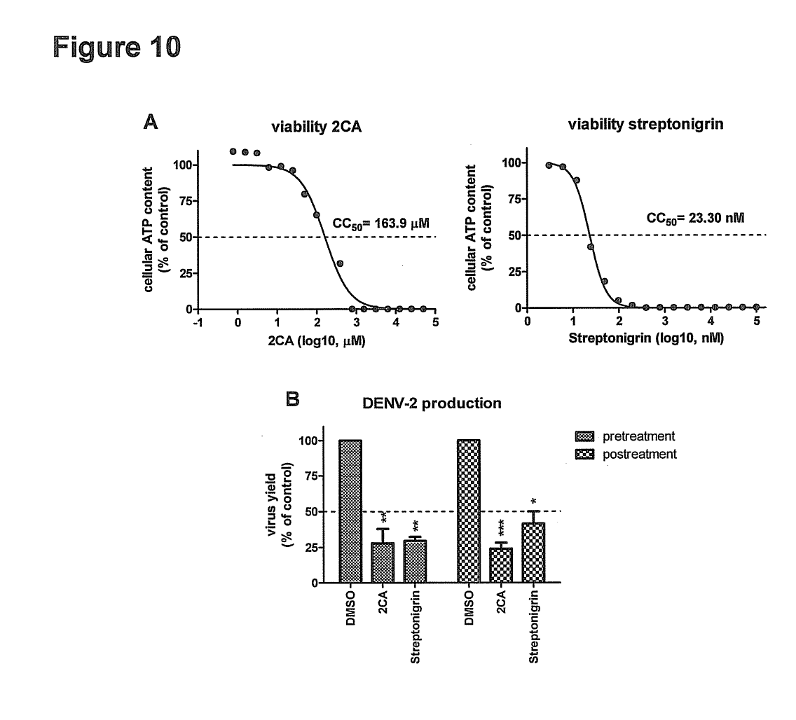

In detail, 71 genes (57 HDFs and 14 HRFs) were identified in Part I and 27 genes (15 HDFs and 12 HRFs) scored specifically only in Part II (FIG. 2 A). Among the HDFs identified in our screen, several components of two different multiple-subunit protein complexes already implicated in the flavivirus replication cycle, the vacuolar ATPase and the proteasome, were identified (FIG. 2 A), validating the reliability of the screening approach (Fernandez-Garcia et al., 2011; Fink et al., 2007; Krishnan et al., 2008; Nag & Finley, 2012; Pattanakitsakul et al., 2007). Out of the remaining hit candidate genes, the majority was so far not linked to DENV or any related flavivirus.

Although hit calling criteria for candidate genes from the primary screen were already stringent, a secondary screen was established to reconfirm the top hit candidates and further enhance reliability and validity of the screen. For the subsequent screen, the hit candidate list form the primary screen was modified to contain only the top scoring genes, fulfilling the even more stringent hit criteria of two siRNAs with a z-score of ≦−2.5 for HDFs or ≧2.5 for HRFs, respectively. In addition, genes from the primary screen, where only a single siRNA scored with a very high z-score (≦−5 for HDFs in part I and ≧4 or ≦−4 for all other siRNAs, respectively) were included in the final validation list to not miss potential interesting hit candidates.

The Secondary screen using the infectious reporter virus DV-R2A was performed twice in duplicates according to the protocol for the Primary siRNA screen but with some important modifications (FIG. 1). Of note: (1) genes were targeted using a set of 4 independent siRNAs per gene obtained from a different supplier to minimize the chances of false positive hits due to off-target effects; (2) the assay format was adapted to 96-well plates, increasing the number of transfected cells and thus the statistical power of the assay. The final consolidated hit candidate list was compiled defining hit calling criteria by a total of three independent siRNAs throughout the primary and the validation screen having z-scores ≦−2 or ≧2, respectively. Using these stringent criteria, a total of 28 HDFs and one HRF could be identified (see FIG. 2 B).

### 2.2 Orthogonal Validation Screen: Discovery of Novel Druggable Host Cell Factors Involved in DENV Replication

HDFs identified by the siRNA-based screen promote viral replication and thus, their inhibition should impair replication of the virus. Since we used a siRNA library covering the human druggable genome, we next determined whether pharmacological inhibition of identified HDFs indeed impairs the DENV life cycle. For 8 of the 29 candidate hits identified after the Secondary screen (and shown in FIG. 2B) well-characterized and commercially available chemical inhibitors or activators were found (see Table 1 below).

The effect of each drug (FIG. 6) on the DENV replication cycle was assessed in Huh7 cells by using a virus yield inhibition assay and two different settings, either with or without pretreatment of the cells with a given drug (FIG. 3 A). Using this approach, the involvement of 4 out of 8 candidate proteins in DENV infection could be validated (FIG. 3 B). Pharmacological modulation of GCKR (glucokinase regulatory protein), PADI4 (peptidyl arginine deiminase, type IV) and PPARS (peroxisome proliferator-activated receptor delta) exhibited the strongest reduction of virus production in the pretreatment conditions, while treatment with an agonist/antagonist of P2X4R (purinergic receptor P2X, ligand-gated ion channel) showed the strongest phenotype in post-treatment conditions (FIG. 3 B).

To determine whether involvement of the four validated genes is unique to DENV or whether viruses from the same and other virus families also utilize these genes for efficient replication, we analyzed the impact of the compounds on the production of infectious HCV (family Flaviviridae) and vesicular stomatitis virus (VSV, family Rhabdoviridae). Interestingly, a similar effect on HCV production was observed upon treatment with the different drugs, albeit to a lesser extent, while there were no effect on the amount of infectious VSV particles released into the supernatant (FIG. 3 C). These results suggest that the identified factors are not only involved in DENV replication cycle, but represent key proteins implicated in the replication of other members within the Flaviviridae family.

TABLE 1

| | | | | | Catalog | Concentra- | | Maximum ef- |
|---|---|---|---|---|---|---|---|---|
| | Name in | | | | no./ | tion used | | fect on DENV |
| Screen target | FIG. 3B | Trivial name | IUPAC name | Description | Provider | (FIG. 3B) | | replication |
| ALDH2 | ALDH2 | Daidzin | Daidzein-7-O-β-D- | Selective inhibitor of | 30408 | 200 | μM | inactive |
| (Aldehyde | inhibitor | | glucopyranoside | ALDH2 | (sigma) | | | |
| dehydrogenase | ALDH2 | Alda-1 | N-(1,3- | Selective enhancer of | 126920 | 80 | μM | inactive |
| 2) | activator | | Benzodioxol-5- | ALDH1 and ALDH2 | (merck) | | | |
| | | | ylmethyl)-2,6- | | | | | |
| | | | dichlorobenzamide | | | | | |
| CHRM2 | CHRM2 | Atropine | α- | Competitive nonselective | A0257 | 100 | μM | inactive |
| (Cholinergic | antagonist 1 | | (Hydroxymethyl)benzene- | antagonist at central and | (sigma) | | | |
| receptor | | | acetic acid 8- | peripheral muscarinic | | | | |
| muscarinic 2) | | | methyl-8- | acetylcholine receptors. | | | | |
| | | | azabicyclo[3.2.1]oct- | | | | | |
| | | | 3-yl ester | | | | | |
| | CHRM2 | Methoctramine | N,N,′-bis[6-[[(2- | Selective M2 muscarinic | M105 | 1 | μM | inactive |
| | antagonist 2 | hydrate | Methoxy- | receptor antagonist at nM | (sigma) | | | |
| | | | phenyl)methyl]amino]hexyl]- | concentrations. At mM | | | | |
| | | | 1,8-octane | concentrations, directly | | | | |
| | | | diaminetetrahydrochloride | inhibits the high affinity | | | | |
| | | | hydrate | GTPase activity of G | | | | |
| | | | | proteins | | | | |
| | CHRM2 | Oxotremorine | N,N,N,-trimethyl-4- | Nonselective muscarinic | O100 | 100 | μM | inactive |
| | agonist | M | (2-oxo-1- | acetylcholine receptor | (sigma) | | | |
| | | | pyrrolidinyl)-2- | agonist. | | | | |
| | | | butyn-1-ammonium | | | | | |
| | | | iodide | | | | | |
| GCKR | GCKR | Cpd A | 2-Amino-5-(4- | A cell-permeable | 346021 | 90 | μM | 67.95% |
| (glucokinase | inhibitor | | metyl-4H-(1,2,4)- | thiazolylamide that | (merck) | | | inhibition |
| regulatory | | | triazole-3-yl- | stabilizes the glucokinase | | | | |
| protein) | | | sulfanyl-N-(4- | in an active conformation | | | | |
| | | | metyl-thiazole-2- | and prevents its interaction | | | | |
| | | | yl)benzamide | with and nuclear | | | | |
| | | | | sequestration by GCKR | | | | |
| GRIK4 | GRIK4 | Methylglutamic | (2S,4R)-4- | Selective and high affinity | G137 | 250 | μM | inactive |
| (glutamate | antagonist | acid | Methylglutamic | kainate receptor antagonist. | (sigma) | | | |
| recptor, | | | acid | | | | | |
| ionotropic | GRIK4 | Kainic acid | 2-Carboxy-3- | Agonist for kainate-class | K2389 | 1 | mM | inactive |
| kainite 4) | agonist | monohydrate | carboxymethyl-4- | ionotropic glutamate | (sigma) | | | |
| | | | isopropenylpyrrolidine | receptors. | | | | |
| P2X4R | P2X4R | TNP-ATP | 2′,3′,-O-(2,4,6- | Purinoceptor P2X | T4193 | 75 | μM | 87.09% |
| (purinergic | antagonist | hydrate | Trinitrophenyl) | antagonist | (sigma) | | | inhibition |
| receptor P2X, | | | adenosine 5′- | | | | | |
| ligand-gated | | | triphosphate | | | | | |
| ion channel 4) | | | monolithiumtrisodium | | | | | |
| | | | salt | | | | | |
| | P2X4R | Bz-ATP | 2′(3′)-O-(4 | Selective P2X purinergic | B6396 | 100 | μM | 46.29% |
| | agonist | | Benzoylbenzoyl)aden- | agonist | (sigma) | | | enhancement |
| | | | osine 5′- | | | | | |
| | | | triphosphate | | | | | |
| | | | triethylammonium | | | | | |
| | | | salt | | | | | |
| PADI4 | PADI4 | Cl-amidine | N-α-benzoyl-N5- | Cell-permeable compound | 506282 | 200 | μM | 92.69% |
| (peptidyl | inhibitor | | (2-chloro-1- | that acts as a pan PADI | (merck) | | | inhibition |
| arginine | | | iminoethyl)-L- | inhibitor | | | | |
| deiminase, | | | ornithine amide | | | | | |
| type IV) | | | | | | | | |
| PPARδ | PPARD | GSK0660 | 3-(((2-Methoxy-4- | Potent PPARβ/δ | G5797 | 10 | μM | 61.68% |
| (peroxisome | antagonist | | (phenyl- | antagonist. GSK0660 is | (sigma) | | | inhibition |
| proliferator- | | | amino)phenyl)amino)sul- | nearly inactive on PPARα | | | | |
| activated | | | fonyl)-2-thiophenecarboxylic | and PPARγ with IC50s | | | | |
| receptor | | | acid methyl ester | greater than 10 μM. | | | | |
| delta) | PPARD | GW0742 | 4-[2-(3-Fluoro-4- | Highly selective PPARδ | G3295 | 1.25 | μM | 47.97% |
| | agonist | | trifluoromethyl- | agonist. EC₅₀ = 1 nM vs 1 | (sigma) | | | enhancement |
| | | | phenyl)-4-methyl- | and 2 mM for PPARα and | | | | |
| | | | thiazol-5- | PPARγ, respectively. | | | | |
| | | | ylmethylsulfanyl]- | | | | | |
| | | | 2-methyl- | | | | | |
| | | | phenoxy}-acetic | | | | | |
| | | | acid | | | | | |
| TRPM8 (also | TRPM8 | Capsazepine | N-[2-(4- | Potent TRPM8 antagonis | C191 | 0.3 | μM | inactive |
| CMR1) | antagonist | | Chlorophenyl)ethyl]- | | (sigma) | | | |
| (transient | | | 1,3,4,5- | | | | | |
| receptor | | | tetrahydro-7,8- | | | | | |
| potential | | | dihydroxy-2H-2- | | | | | |
| cation | | | benzazepine-2- | | | | | |
| channel 8) | | | carbothioamide | | | | | |
| | TRPM8 | Menthol | 2-Isopropyl-5- | Specific agonist of TRPM8 | M2772 | 250 | μM | inactive |
| | agonist 1 | | methylcyclohexanol | | (sigma) | | | |
| | TRPM8 | WS-12 | (2S,5R)-2- | high-affinity, selective | W0519 | 50 | μM | inactive |

| | | | |
|---|---|---|---|
| agonist 2 | Isopropyl-N-(4- | TRPM8 agonist. | (sigma) |
| | methoxyphenyl)-5- | | |
| | methylcyclohexane | | |
| | carboximide | | |

### 2.3 PADI4 as a Novel Target to Prevent DENV Infection

Among the factors validated in the orthogonal screen, inhibition of PADI4 showed the strongest phenotype on DENV production (FIG. 3 B).

The compound N-α-benzoyl-N5-(2-chloro-1-iminoethyl)-L-ornithine amide (Cl-amidine) is a cell-permeable pan PADI inhibitor.

We determined the potency of Cl-amidine against DENV-2 infection (median effective concentration, EC₅₀) and its toxicity (median cytotoxic concentration, CC₅₀) in Huh7 cells. Treatment with serial dilutions of Cl-amidine resulted in a strong dose-dependent reduction of virus production, reaching up to 100-fold inhibition of virus titers at the highest concentration tested (FIG. 4 A). Of note, the observed reduction in virus production is not due to a negative impact on cell viability, since Cl-amidine did not produce cytotoxic effect in Huh7 cells up to 1000 μM concentration (FIG. 4 B). This yields a selectivity index (SI: CC₅₀/EC₅₀) for Cl-amidine >62.15.

In order to corroborate that the inhibitory effect exerted by Cl-amidine is due to its effect targeting PADI4, we generated a Huh7 cell line stably overexpressing PADI4 (Huh7-PADI4). Using the same methodology, a control cell line was established carrying an empty expression vector (Huh7-Empty). Efficient overexpression of the transgene was confirmed by indirect immunofluorescence assay using specific PADI4 antibodies, which revealed an accumulation of the protein in the nucleus (FIG. 4 C). Virus yield inhibition assays performed in these cell lines showed that overexpression of PADI4 reduced the impact of Cl-amidine on virus production, as deduced from the around 10-fold higher EC₅₀ as compared to Huh7-Empty cells (FIG. 1 A and Table 2I). These results demonstrate that PADI4 is required for DENV infection and that this host factor can be specifically targeted by Cl-amidine.

TABLE 2
Antiviral activity and specificity of Cl-amidine.

| Cell line | EC₅₀ ± SD (μM) | EC₅₀ shift (fold) |
|---|---|---|
| Parental Huh7 | 16.09 ± 4.30 | — |
| Huh7-Empty | 19.34 ± 2.36 | 1 |
| Huh7-PADI4 | 215.35 ± 18.98 | 11.13 |

Identification of host factors using RNAi may depend to a large extent on the cellular system chosen to perform the screen, what argues for the need of testing the selected hit candidates in cellular systems more representative of the infection in nature. Therefore, primary human monocytes were isolated foul′ peripheral blood of three healthy donors and infected with DENV-2 in the presence of serial dilutions of Cl-amidine. The impact of the drug on virus production was assessed by plaque assay 48 h after infection. In this condition, Cl-amidine showed a dose-dependent inhibitory effect similar to that observed in Huh7 cells, with EC₅₀ values ranging between ˜17 μM and ˜25 μM among different donors (FIG. 5 A and Table 3). Importantly, in the conditions of the assay Cl-amidine was not cytotoxic for primary human monocytes (FIG. 5 B).

TABLE 3
Effect of Cl-amidine in primary cells.

| Origin of monocytes | EC₅₀ (μM) |
|---|---|
| Donor 1 | 16.95 |
| Donor 2 | 24.61 |
| Donor 3 | 22.95 |

Given the only 62-67% of amino acid conservation among DENV serotypes (Kyle & Harris 2008), the different serotypes might not be equally dependent on the same host factors for their replication. Therefore, we evaluated the impact of Cl-amidine (200 μM) on the production of DENV-1, -3 and -4 in parallel to DENV-2 in Huh7 cells using the experimental conditions previously established for DENV-2. A similar inhibitory effect was observed for all the four DENV serotypes, reaching 10-fold reduction on virus production (FIG. 5 C).

### Example 2

### 1. Materials and Methods

### 1.1 Cells and Viruses

The human hepatocarcinoma cell line Huh7, monkey kidney VeroE6, Baby hamster kidney BHK and HEK293T cells were cultivated in Dulbecco's modified minimal essential medium (DMEM; Life Technologies, Frankfurt, Germany) supplemented with 2 mM L-glutamine, non-essential amino acids, 100 U/ml penicillin, 100 μg/ml streptomycin, and 10% fetal calf serum (DMEMcplt).

The Renilla luciferase reporter DENV-2 16681 (DV-R2A) which encodes a Renilla luciferase was described elsewhere (Fischl and Bartenschlager 2013). Virus stocks (DV-R2A or DENV wild type) were generated by transfection of in vitro transcribed viral RNAs into BHK cells by electroporation (seed stocks) followed by one round of amplification in Huh7 cells (Fischl and Bartenschlager 2013).

### 1.2 Antibodies and Reagents

The rabbit polyclonal anti-PADI4 (ab50247) was purchased from Abcam. Cl-amidine (506282) was obtained from Merck. Chloroacetamidine (2CA) (591475) and streptonigrin (S1014) were purchased from Sigma-Aldrich. Chemical inhibitors were reconstituted in DMSO, aliquoted and stored at −20° C. until used.

### 1.3 Cell Viability and Virus Yield Reduction Assays

The suitable concentration of each chemical compound to be tested in virus yield inhibition assays was first established by determining the effect of each drug on cell viability using a bioluminescent method that allows quantification of ATP content within cells (CellTiter-Glo® Luminescent Cell Viability Assay kit, Promega), following manufacturer instructions. Viability was determined as the ratio between signals in treated cultures vs vehicle control cells. Concentrations exhibiting 80% cell viability or higher by both methods were chosen for subsequent assays.

The effect of each compound on virus production was assessed by a virus yield reduction assay using DENV-2 wild type virus. Huh7 cells seeded in 24-well plates were either pretreated or not for 2 h with non-cytotoxic concentrations of the drugs and then infected with DENV-2 in presence or absence of the compounds or vehicle at a MOI of 0.1 PFU/cell. After 1 h incubation at 37° C. viral inocula were removed and replaced by DMEMcplt containing the drugs. Virus titers were determined after 48 h by plaque assay in VeroE6 cells using 1.5% carboxymethylcellulose plaquing medium.

### 1.4 In Vitro Transcription and RNA Transfection.

In vitro transcripts were generated as previously described (Fischl and Bartenschlager 2013). For RNA transfection, Huh7 cell suspensions were prepared by trypsinization, washed with PBS, and resuspended in Cytomix (supplemented with 2 mM ATP and 5 mM glutathione) at a concentration of 1×10⁷ cells/ml. Next, 10 μg of subgenomic or genomic in vitro transcript was mixed with 400 μl of the cell suspension and transfected by electroporation using a Gene Pulser system (Bio-Rad) and a cuvette with a gap width of 0.4 cm (Bio-Rad) at 975 μF and 270 V. Cells were immediately diluted into 10 ml of DMEM cplt and seeded in the appropriate format.

### 1.5 Lentiviral Particle Production and Cell Transduction

Recombinant lentiviral vector viruses were generated by calcium phosphate mediated co-transfection of a lentiviral vector plasmid along with two packaging plasmids into HEK293T cells using the CalPhos™ Mammalian Transfection kit. Briefly, 1.2×10⁶ HEK293Tcells plated onto 6 cm dishes were transfected with, 6.4 μg gag-pol plasmid (pCMV-dR8.91), 2.1 μg VSV-G envelope expression plasmid (pMD.G) and 6.4 μg of each of the pLKO.1-based shRNA vector (Mission shRNA, Sigma-Aldrich, Table 4). After 6-16 hours, the medium was replaced with 5 ml of fresh DMEMcplt and 24 h later supernatants containing infectious lentiviral particles, were harvested, filtrated using a 0.45-μm syringe tip filter, aliquoted and stored at −80° C. until use. For transient silencing, Huh7 cells were transduced in suspension with each lentivirus at a MOI of 5 transforming units (TU)/ml in the presence of Polybrene (4 μg/ml).

TABLE 4
Sequences of shRNAs

| shRNA | |
|---|---|
| [SEQ ID NO.] | Sequence |
| shNT#1 | CCGGCAACAAGATGAAGAGCACCAACTCGA |
| [21] | GTTGGTGCTCTTCATCTTGTTGTTTTT |
| shNT#2 | CCGGCAACAAGATGAAGAGCACCAACTCGA |
| [22] | GTTGGTGCTCTTCATCTTGTTGTTTTT |
| shPADI4#1 | CCGGCTGAAGGAGTTTCCCATCAAACTCGA |
| [23] | GTTTGATGGGAAACTCCTTCAGTTTTTG |
| shPADI4#2 | CCGGAGACATTGAGAGAACATAATTCTCGA |
| [24] | GAATTATGTTCTCTCAATGTCTTTTTTTG |
| shPADI4#3 | CCGGTGACTACTCTGGCCATGAAAGCTCGA |
| [25] | GCTTTCATGGCCAGAGTAGTCATTTTTTG |
| shPADI4#4 | CCGGCCAGGTCTGAGATGGACAAAGCTCGA |
| [26] | GCTTTGTCCATCTCAGACCTGGTTTTTTG |
| shPADI4#5 | CCGGAGCAAGAGCTCTTGTGAATATCTCGA |
| [27] | GATATTCACAAGAGCTCTTGCTTTTTTTG |

### 1.6 Generation of DENV Trans-Complemented Particles (DENV_{TCP})

Single-round infectious particles were produced by trans-complementation in HEK293T cells stably expressing DENV prM-E proteins as previously described (Scaturro et al. 2014). Briefly, 8×10⁶ cells were electroporated with 10 μg of subgenomic Renilla-reporter replicon RNA, seeded into 15 cm-diameter dishes and incubated at 33° C. On the next day, cells were inoculated with lentiviral particles encoding for C and prM-E proteins. After 8 h cells were carefully washed 3 times with PBS, covered with fresh DMEMcplt containing 15 mM HEPES (4-(2-Hydroxyethyl)piperazine-1-ethanesulfonic acid) and cultured at 33° C. for 4-6 days. Supernatants were harvested, filtered through a 0.45 μm-pore-size filter, aliquoted and stored at −80° C. until use.

### 1.7 Confocal Immunofluorescence Microscopy

Huh7 cells seeded on coverslips were infected with DENV-2 at a moi of 1 PFU/cell. Fixation was performed after 48 h of infection and detection of endogenous PADI4 was performed using a rabbit polyclonal antiserum against PADI4 (Abcam) followed by incubation with a secondary antibody coupled to Alexa-Fluor488. Cells were mounted on microscope glass slides using Fluoromount G and images were acquired using a Nikon microscope.

### 2. Results

2.1 the Susceptibility of DENV to Cl-Amidine Treatment is Enhanced in Cells with Reduced Expression of PADI4

In order to corroborate that Cl-amidine-mediated inhibition of DENV infection is due to the specific effect of this drug targeting PADI4, we performed a sensitization assay combining Cl-amidine treatment with shRNA-mediated gene know-down. In first place, we generated PADI4-knockdown cell populations by transducing Huh7 cells with lentiviral vectors expressing five different shRNAs against PADI4 mRNA or irrelevant sequences (shNT) used as a control. Of note, the sequences used to target PADI4 were different from those used in the siRNA screens, reducing the chances that the observed effect on virus production are simply due to off target effects. Cell populations were infected with DV-R2A 48 h after gene silencing and virus replication was assessed 48 h after infection by measuring Renilla luciferase (R-Luc) activity from cell lysates. As seen in FIG. 7 A, all shRNAs targeting PADI4 produced a moderate, although significant reduction in DENV replication. To test whether the decrease replication observed correlates with a diminution of infectious particle production, we measured the viral titer of culture media collected at the end point of the assay. In agreement with the replication data, a significant reduction in DENV production was observed (FIG. 7 B) that correlates with the impact of the shRNAs on virus replication (FIG. 7 A). Of note, the inhibitory effect was not due to shRNAs-induced cytotoxicity (FIG. 7 B). Then, we investigated whether reducing the levels of PADI4 in combination with a specific inhibitor of this protein (Cl-amidine) would induce a synergistic inhibitory effect on virus production. Huh7 cells were transduced with lentiviral vectors harboring shPADI4#4 or shNT#2 and 48 h later virus yield reduction assays were performed using various concentrations of Cl-amidine. As expected, the EC₅₀ value in cells transduced with shNT#2 was similar to that obtained in parental Huh7 cells (EC_{50-shNT#2}: 17,11 μM), while silencing of PADI4 produced a shift in the EC₅₀ value of approximately 14-fold towards lower concentrations (EC_{50-shpADI#4}: 1.18 μM) (FIG. 7 D). Importantly, we verified that the enhanced inhibitory effect of the drug upon gene silencing does not result from an additive cytotoxic effect (FIG. 7 E). Taken together these results further reinforce our previous finding (see Example 1) that PADI4 is required for efficient DENV production and demonstrate that Cl-amidine-mediated inhibition of DENV infection is not due to a pleiotropic effect induced by this drug.

### 2.2 PADI4 is Required at the Onset of DENV Genome Replication

In order to determine the step in DENV life cycle at which PADI4 is involved, we performed time-of-addition experiments using defective DENV particles that are able to undergo only one single round of infection (denominated DENV-transcomplemented particles, DENV_{TCP}). These particles were produced by transfecting a subgenomic DENV Renilla-reporter replicon (DENV_{sg}-R2A) lacking the C-prM-E coding sequences into packaging cells that provide the DENV structural proteins in trans (Scaturro, et al 2014). Huh7 cells were infected with DENV_{TCP} and Cl-amidine (200 μM) was added 2 h before infection, during inoculation or at different time points post-infection. Virus replication was scored 48 h later by measuring R-luc counts in cell lysates. We observed a maximal reduction in virus replication when Cl-amidine was added before infection or at early time points after infection, and this inhibitory effect remained significant when the drug was added up to 8 h after infection (FIG. 8 A). This decreased replication upon Cl-amidine addition indicates that PADI4 may be involved in virus entry, translation or replication of incoming virus genomes.

To determine whether Cl-amidine treatment affected the viral entry process or a post-entry event, we bypassed the stages from virus attachment to membrane fusion by directly delivering viral RNA molecules into the cell cytoplasm. Viral RNA derived from DENV_{sg}-R2A were generated by in vitro transcription and introduced into Huh7 cells by electroporation. Cells were immediately treated with increasing concentrations of Cl-amidine and 48 h later cultures were lysed and the level of replication was determined by measuring R-Luc activity. In these conditions, Cl-amidine significantly reduced DENV-driven luciferase activity to levels similar to those achieved in virus yield inhibition assays, exhibiting an EC₅₀ value of 16.52±2.49 μM (FIG. 8 B). This result clearly demonstrates that PADI4 is involved in a post-entry step of DENV life cycle.

In order to discriminate whether Cl-amidine treatment impacts the initiation of transcription or primary translation of viral genomes, we performed kinetics studies to measure renilla accumulation in cells electroporated with subgenomic RNA molecules bearing or not a replication defective mutation in NS5 (DENV(GND)_{sg}-R2A and DENV_{sg}-R2A, respectively). While accumulation of R-Luc counts in cells electroporated with DENV_{sg}-R2A RNA can be used as a surrogate marker for translation and replication, the GND point mutation in NS5 renders the polymerase inactive, and thus, luciferase activity detected form cells lysates is a reflect of primary translation from input RNA molecules. Huh7 cells were electroporated with equal amounts of each in vitro transcript, treated with Cl-amidine (200 μM) and lysed at different time points after transfection. We observed no difference in luciferase activity during the initial 10 h of transfection between Cl-amidine- and DMSO-treated cells for both _{sg}DENV-R2A and DENV(GND)_{sg}R2A RNA species, indicating that PADI4 is not required for primary translation of incoming RNA molecules (FIG. 8 C). However, a circa 10-fold reduction was detected at later time points in Cl-amidine-treated cells electroporated with DENV_{sg}-R2A transcripts (FIG. 8 C), indicating that PADI4 is required for the synthesis of DENV genomes.

Our single round of infection time-of-addition experiment demonstrates that Cl-amidine does not restrict DENV replication when added at later time points (FIG. 8 A), suggesting that inhibition of PADI4 would not interfere with stady-state DENV replication. To test this hypothesis we evaluated the effect of Cl-amidine on virus replication in Huh7 cell pools harboring a Hygromycin B-selectable reporter subgenomic replicon (designated as DENV_{sg}-R2H) in parallel to Huh7 cells freshly transfected with DENV_{sg}-R2A RNA molecules. As expected, the presence of 200 μM Cl-amidine reduced virus replication in freshly electroporated cells by 10-fold compared to vehicle-treated cells. However, in stable replicon cells the presence of the drug reduced R-Luc activity by only 1.5-fold (FIG. 8 D).

Overall, these results indicate that PADI4 is not required for translation of incoming RNA molecules but is a rate-limiting factor at the early steps of infection that leads to viral RNA accumulation.

### 2.3 PADI4 is Redistributed Upon DENV Infection

Next, we performed immunofluorescence staining in DENV-infected and uninfected Huh7 cells to investigate whether DENV infection would have an impact on the subcellular localization of endogenous PADI4. PADI4 is the only PADI family member containing a nuclear localization signal and it was shown to citrullinate many nuclear substrates including histones H3, H2A, and H4, p300/CREB-binding protein, nucleophosmin, ING4, and nuclear lamin C to exert various functions (Lee et al 2005; Wang et al 2004; Zhang et al 2011). In agreement, we found that in mock-infected Huh7 cells PADI4 is manly located in discrete nuclear punctate structures (FIG. 9 A). This nucleolar distribution of PADI4 has been described previously (Wang et al. 2012), and is in line with previous findings revealing that PADI4 is able to citrullinate proteins located in the nucleoulus, such as the histone chaperon nucleophosmin. Infection with DENV-2 drastically altered the distribution of PADI4 from nucleolar to a diffuse nuceloplasmic and cytoplasmic pattern (FIG. 9 A). This observation was confirmed by quantification of signal intensities from 50 cells (FIG. 9 B). These observations indicate that in DENV-infected cells PADI4 is relocalized to the cytoplasm, presumably at sites where DENV replication takes place.

2.4 Targeting PADI4 with 2-Chloroacetamidine and Streptonigrin Reduce DENV Production.

In addition to Cl-amidine, other molecules inhibiting PADI4 enzymatic activity have been reported. 2-Chloroacetaminine (2CA), the warhead in Cl-amidine, is an irreversible, time- and concentration dependent, active-site-directed inactivator of PAD4 in vitro (Stone et al 2005), and was shown to reverse protein-hypercitrullination and disease in mouse models of multiple sclerosis (Moscarello et al 2013). More recently, streptonigrin a molecule with reported anti-tumor and anti-bacterial activity, was identified as an effective PAD4 inhibitor acting at nanomolar range (Knuckley et al 2010-2). In particular, the 7-amino-quinoline-5,8-dione core of streptonigrin was identified as a highly potent pharmacophore that acts as a pan-PADI inhibitor (Dreyton et al 2014).

Based on our findings of the dependence on PADI4 for DENV replication we evaluated whether these two inhibitors are able to reduce DENV production in cell culture. In first place we established the maximal concentrations of each compound that can be applied to cells without inducing considerable cytotoxicity (FIG. 10 A). Then we evaluated their antiviral activity in Huh7 cultures in conditions of pretreatment or treatment only after DENV infection. We observed that both, 2CA and streptonigrin, exert significant inhibitory effect on virus production: treatment with 50 μM of 2CA yielded a 77% inhibition on virus production while treatment with 12 nM streptonigrin reduced virus production by 78% (FIG. 10 B).

These demonstrate that inhibition of DENV production can be achieved using diverse molecules that share in common their ability to target PADI4 activity.

The features disclosed in the foregoing description, in the claims and/or in the accompanying drawings may, both separately and in any combination thereof, be material for realizing the invention in diverse forms thereof.

### REFERENCES

Acosta E G, Castilla V, Damonte E B (2009) Alternative infectious entry pathways for dengue virus serotypes into mammalian cells. Cellular Microbiology 11: 1533-1549.
Arita K, Hashimoto H, Shimizu T, Nakashima K, Yamada M, Sato M. (2004) Structural basis for Ca(2+)-induced activation of human PAD4. Nat Struct Mol Biol. 11(8):777-83.
Bhatt S, Gething P W, Brady O J, Messina J P, Farlow A W, Moyes C L, Drake J M, Brownstein J S, Hoen A G, Sankoh O, Myers M F, George D B, Jaenisch T, Wint G R, Simmons C P, Scott T W, Farrar J J, Hay S I. (2013) The global distribution and burden of dengue. Nature 496(7446):504-7.
Bicker K L, Thompson P R. (2013) The protein arginine deiminases: Structure, function, inhibition, and disease. Biopolymers. 99(2):155-63.
Boutros M, Bras L P, Huber W (2006) Analysis of cell-based RNAi screens. Genome Biol 7: R66.
Brideau C, Gunter B, Pikounis B, Liaw A (2003) Improved statistical methods for hit selection in highthroughput screening. J Biomol Screen 8: 634-647.
Christophorou M A, Castelo-Branco G, Halley-Stott R P, Oliveira C S, Loos R, Radzisheuskaya A, Mowen K A, Bertone P, Silva J C, Zernicka-Goetz M, Nielsen M L, Gurdon J B, Kouzarides T (2014) Citrullination regulates pluripotency and histone H1 binding to chromatin. Nature. 507(7490):104-8.
Chumanevich A A, Causey C P, Knuckley B A, Jones J E, Poudyal D, Chumanevich A P, Davis T, Matesic L E, Thompson P R, Hofseth L J. (2011) Suppression of colitis in mice by Cl-amidine: a novel peptidylarginine deiminase inhibitor. Am J Physiol Gastrointest Liver Physiol. 300(6):G929-38.
Darrah E, Rosen A, Giles J T, Andrade F. (2012) Peptidylarginine deiminase 2, 3 and 4 have distinct specificities against cellular substrates: novel insights into autoantigen selection in rheumatoid arthritis. Ann Rheum Dis. 71(1):92-8
Dreyton C J, Anderson E D, Subramanian V, Boger D L, Thompson P R. (2014) Insights into the mechanism of streptonigrin-induced protein arginine deiminase inactivation. Bioorg Med Chem. 22:1362-9.
Fernandez-Garcia, M. D., Meertens, L., Bonazzi, M., Cossart, P., Arenzana-Seisdedos, F., & Amara, A. (2011). Appraising the roles of CBLL1 and the ubiquitin/proteasome system for flavivirus entry and replication. J Virol, 85(6), 2980-2989.
Fink, J., Gu, F., Ling, L., Tolfvenstam, T., Olfat, F., Chin, K. C., et al. (2007). Host gene expression profiling of dengue virus infection in cell lines and patients. PLoS Negl Trop Dis, 1(2), e86.
Fischl W, Bartenschlager R (2013) High-throughput screening using dengue virus reporter genomes. Methods Mol Biol. 1030:205-19.
Folly B B, Weffort-Santos A M, Fathman C G, Soares L R (2011) Dengue-2 structural proteins associate with human proteins to produce a coagulation and innate immune response biased interactome. BMC Infect Dis 11: 34.
Gastaminza P, Whitten-Bauer C, Chisari F V. (2010) Unbiased probing of the entire hepatitis C virus life cycle identifies clinical compounds that target multiple aspects of the infection. Proc Natl Acad Sci USA. 107(1):291-6.
Gubler D J (1998) Dengue and dengue hemorrhagic fever. Clinical Microbiology Reviews 11: 480-96
Halstead S B (2007) Dengue. Lancet. 370(9599):1644-52.
Jessie, K., Fong, M. Y., Devi, S., Lam, S. K., & Wong, K. T. (2004). Localization of dengue virus in naturally infected human tissues, by immunohistochemistry and in situ hybridization. J Infect Dis, 189(8), 1411-1418.
Knight J S, Zhao W, Luo W, Subramanian V, O'Dell A A, Yalavarthi S, Hodgin J B, Eitzman D T, Thompson P R, Kaplan M J. (2013) Peptidylarginine deiminase inhibition is immunomodulatory and vasculoprotective in murine lupus. J Clin Invest 123(7):2981-93.
Knight J S, Luo W, O'Dell A A, Yalavarthi S, Zhao W, Subramanian V, Guo C, Grenn R C, Thompson P R, Eitzman D T, Kaplan M J. (2014) Peptidylarginine Deiminase Inhibition Reduces Vascular Damage and Modulates Innate Immune Responses in Murine Models of Atherosclerosis. Circ Res. 14; 114(6):947-56
Knucldey B, Causey C P, Jones J E, Bhatia M, Dreyton C J, Osborne T C, Takahara H, Thompson P R. (2010) Substrate specificity and kinetic studies of PADs 1, 3, and 4 identify potent and selective inhibitors of protein arginine deiminase 3. Biochemistry. 49(23):4852-63.
Knuckley B, Jones J E, Bachovchin D A, Slack J, Causey C P, Brown S J, Rosen H, Cravatt B F, Thompson P R. (2010-2) A fluopol-ABPP HTS assay to identify PAD inhibitors. Chem Commun (Camb). 46:7175-7.
Kolodziej S, Kuvardina O N, Oellerich T, Herglotz J, Backert I, Kohrs N, Buscató El, Wittmann S K, Salinas-Riester G, Bonig H, Karas M, Serve H, Proschak E, Lausen J. (2014) PADI4 acts as a coactivator of Tall by counteracting repressive histone arginine methylation. Nat Commun. 5:3995.
Krishnan, M. N., Ng, A., Sukumaran, B., Gilfoy, F. D., Uchil, P. D., Sultana, H., et al. (2008). RNA interference screen for human genes associated with West Nile virus infection. Nature, 455(7210), 242-245.
Kyle J L & Harris E. (2008) Global spread and persistence of dengue. Annu Rev Microbiol. 62:71-92.
Lee Y H, Coonrod S A, Kraus W L, Jelinek M A, Stallcup M R. (2005) Regulation of coactivator complex assembly and function by protein arginine methylation and demethylimination. Proc Natl Acad Sci USA. 102(10):3611-6.
Limon-Flores, A. Y., Perez-Tapia, M., Estrada-Garcia, I., Vaughan, G., Escobar-Gutierrez, A., Calderon-Amador, J., et al. (2005). Dengue virus inoculation to human skin explants: an effective approach to assess in situ the early infection and the effects on cutaneous dendritic cells. Int J Exp Pathol, 86(5), 323-334.
Luo Y, Arita K, Bhatia M, Knuckley B, Lee Y H, Stallcup M R, Sato M, Thompson P R. (2006) Inhibitors and inactivators of protein arginine deiminase 4: functional and structural characterization. Biochemistry. 45(39):11727-36.
Manzoor S, Idrees M, Ashraf J, Mehmood A, Butt S, Fatima K, Akbar H, Rehaman I U, Qadri I. (2011) Identification of ionotrophic purinergic receptors in Huh-7 cells and their response towards structural proteins of HCV genotype 3a. Virol J. 8:431.
Martina B E, Koraka P, Osterhaus A D. (2009) Dengue virus pathogenesis: an integrated view. Clin Microbiol Rev. 22(4):564-81.
Martinod K, Demers M, Fuchs T A, Wong S L, Brill A, Gallant M, Hu J, Wang Y, Wagner D D. (2013) Neutrophil histone modification by peptidylarginine deiminase 4 is critical for deep vein thrombosis in mice. Proc Natl Acad Sci USA. 110(21):8674-9.
McElwee J L, Mohanan S, Griffith O L, Breuer H C, Anguish L J, Cherrington B D, Palmer A M, Howe L R, Subramanian V, Causey C P, Thompson P R, Gray J W, Coonrod S A. (2012) Identification of PADI2 as a potential breast cancer biomarker and therapeutic target. BMC Cancer. 12:500.
Moscarello M A, Lei H, Mastronardi F G, Winer S, Tsui H, Li Z, Ackerley C, Zhang L, Raijmakers R, Wood D D. (2013) Inhibition of peptidyl-arginine deiminases reverses protein-hypercitrullination and disease in mouse models of multiple sclerosis. Dis Model Mech.; 6:467-78.
Nag, D. K., & Finley, D. (2012). A small-molecule inhibitor of deubiquitinating enzyme USP14 inhibits Dengue virus replication. Virus Res, 165(1), 103-106.
Nakashima K, Arai S, Suzuki A, Nariai Y, Urano T, Nakayama M, Ohara O, Yamamura K, Yamamoto K, Miyazaki T. (2013) PAD4 regulates proliferation of multipotent haematopoietic cells by controlling c-myc expression. Nat Commun. 4:1836.
Pattanakitsakul, S. N., Rungrojcharoenkit, K., Kanlaya, R., Sinchaikul, S., Noisakran, S., Chen, S. T., et al. (2007). Proteomic analysis of host responses in HepG2 cells during dengue virus infection. J Proteome Res, 6(12), 4592-4600.
Rieber N, Knapp B, Eils R, Kaderali L (2009) RNAither, an automated pipeline for the statistical analysis of high-throughput RNAi screens. Bioinformatics 25: 678-679.
Scaturro P, Trist I M, Paul D, Kumar A, Acosta E G, Byrd C M, Jordan R, Brancale A, Bartenschlager R. (2014) Characterization of the mode of action of a potent dengue virus capsid inhibitor. J Virol. 88:11540-55.
Seneviratne, S. L., Malavige, G. N., & de Silva, H. J. (2006). Pathogenesis of liver involvement during dengue viral infections. Trans R Soc Trop Med Hyg, 100(7), 608-614.
St John A L, Abraham S N, Gubler D J. (2013) Barriers to preclinical investigations of anti-dengue immunity and dengue pathogenesis. Nat Rev Microbiol. 11(6):420-6.
Stone E M, Schaller T H, Bianchi H, Person M D, Fast W. (2005) Inactivation of two diverse enzymes in the amidinotransferase superfamily by 2-chloroacetamidine: dimethylargininase and peptidylarginine deiminase. Biochemistry. 44:13744-52.
Vossenaar E R, Zendman A J, van Venrooij W J, Pruijn G J. (2003) PAD, a growing family of citrullinating enzymes: genes, features and involvement in disease. Bioessays. 25(11):1106-18.
Wang S, Wang Y. (2013) Peptidylarginine deiminases in citrullination, gene regulation, health and pathogenesis. Biochim Biophys Acta. 1829(10):1126-35.
Wang Y, Wysocka J, Sayegh J, Lee Y H, Perlin J R, Leonelli L, Sonbuchner L S, McDonald C H, Cook R G, Dou Y, Roeder R G, Clarke S, Stallcup M R, Allis C D, Coonrod S A. (2004) Human PAD4 regulates histone arginine methylation levels via demethylimination. Science 306(5694): 279-83.
Wang Y, Li P, Wang S, Hu J, Chen X A, Wu J, Fisher M, Oshaben K, Zhao N, Gu Y, Wang D, Chen G, Wang Y. (2012) Anticancer peptidylarginine deiminase (PAD) inhibitors regulate the autophagy flux and the mammalian target of rapamycin complex 1 activity. J Biol Chem. 287:25941-53.
Willis V C, Gizinski A M, Banda N K, Causey C P, Knuckley B, Cordova K N, Luo Y, Levitt B, Glogowska M, Chandra P, Kulik L, Robinson W H, Arend W P, Thompson P R, Holers V M. (2011) N-α-benzoyl-N5-(2-chloro-1-iminoethyl)-L-ornithine amide, a protein arginine deiminase inhibitor, reduces the severity of murine collagen-induced arthritis. J Immunol. 186(7):4396-404.
Wu, S. J., Grouard-Vogel, G., Sun, W., Mascola, J. R., Brachtel, E., Putvatana, R., et al. (2000). Human skin Langerhans cells are targets of dengue virus infection. Nat Med, 6(7), 816-820.
Zhang X, Gamble M J, Stadler S, Cherrington B D, Causey C P, Thompson P R, Roberson M S, Kraus W L, Coonrod S A. (2011) Genome-wide analysis reveals PADI4 cooperates with Elk-1 to activate c-Fos expression in breast cancer cells. PLoS Genet. 7(6):e1002112.

1-15. (canceled)

16. A method for the prevention and/or treatment of an infection with a virus of the Flaviviridae family, comprising:
administering to a patient in need thereof at least one inhibitor or antagonist of PADI4 (peptidyl arginine deiminase, type IV), PPARδ (peroxisome proliferator-activated receptor delta), GCKR (glucokinase regulatory protein) and/or P2X4R (purinergic receptor P2X, ligand-gated ion channel 4) or
administering to the patient a pharmaceutical composition, said pharmaceutical composition comprising
at least one inhibitor or antagonist of PADI4, PPARδ, GCKR and/or P2X4R,
optionally, a pharmaceutical excipient, and
optionally, a further antiviral agent.

17. The method of claim 16, for treating one or more viruses of the Flaviviridae family selected from Dengue virus (DENV), Hepatitis C virus (HCV), Yellow fever virus, West Nile virus, Japanese encephalitis virus and Tick-borne encephalitis virus.

18. The method of claim 17, wherein the Dengue virus comprises the four serotypes DENV-1, DENV-2, DENV-3 and DENV-4.

19. The method of claim 1, wherein the inhibitor or antagonist is selected from
N-α-benzoyl-N5-(2-chloro-1-iminoethyl)-L-ornithine amide (Cl-amidine);
3-(((2-Methoxy-4-(phenylamino)phenyl)amino)sulfonyl)-2-thiophene-carboxylic acid methyl ester (GSK0660);
2-Amino-5-(4-methyl-4H-(1,2,4)-triazole-3-yl-sulfanyl-N-(4-methyl-thiazole-2-yl)benzamide (CpdA);
2′,3′,-O-(2,4,6-Trinitrophenyl) adenosine 5′-triphosphate monolithium-trisodium salt (TNP-ATP);
2-chloroethanimidamide hydrochloride (2-Chloroacetamidine hydrochloride, 2CA);
or
(4Z)-5-amino-6-(7-amino-6-methoxy-5,8-dioxoquinolin-2-yl)-4-(4,5-dimethoxy-6-oxocyclohexa-2,4-dien-1-ylidene)-3-methyl-1H-pyridine-2-carboxylic acid (Streptonigrin).

20. The method of claim 16, wherein the inhibitor or antagonist or pharmaceutical composition is administered by one or more of inhalation, intranasal, intravenous, oral, transdermal, sustained release, controlled release, delayed release, suppository, or sublingual administration.

21. The method of claim 16, wherein the inhibitor or antagonist or pharmaceutical composition is administered to a subject in need thereof in combination with a further antiviral agent.

22. A method of screening for antiviral agent(s), selected from:
(A) a method comprising:
(a) adding a compound to be screened to a PADI4, PPARδ, GCKR and/or P2X4R test system;
(b) infecting said test system with a virus of the Flaviviridae family in the presence of said compound to be screened;
(c) removing the viral inoculum and adding said compound to be screened;
(d) quantifying virus production; and
(e) comparing virus production in step (d) with virus production in the absence of the candidate compound, wherein a difference between the measured virus productions indicates that the candidate compound is a modulator of PADI4, PPARδ, GCKR and/or P2X4R, and wherein a decrease in the measured virus production in step (d) indicates that the candidate compound is an inhibitor or antagonist of PADI4, PPARδ, GCKR and/or P2X4R and is, thus, an antiviral agent and
(B) a method comprising:
(a) infecting a PADI4, PPARδ, GCKR and/or P2X4R test system with a virus of the Flaviviridae family;
(b) removing the viral inoculum and adding a compound to be screened;
(c) quantifying virus production; and
(d) comparing virus production in step (c) with the virus production in the absence of the candidate compound, wherein a difference between the measured virus productions indicates that the candidate compound is a modulator of PADI4, PPARδ, GCKR and/or P2X4R, and wherein a decrease in the measured virus production in step (c) indicates that the candidate compound is an inhibitor or antagonist of PADI4, PPARδ, GCKR and/or P2X4R and is, thus, an antiviral agent.

23. The method of claim 22, wherein said method comprises:
(a) infecting a PADI4, PPARδ, GCKR and/or P2X4R test system with a virus of the Flaviviridae family;
(b) removing the viral inoculum and adding a compound to be screened;
(c) quantifying virus production; and
(d) comparing virus production in step (c) with the virus production in the absence of the candidate compound, wherein a difference between the measured virus productions indicates that the candidate compound is a modulator of PADI4, PPARδ, GCKR and/or P2X4R, and wherein a decrease in the measured virus production in step (c) indicates that the candidate compound is an inhibitor or antagonist of PADI4, PPARδ, GCKR and/or P2X4R and is, thus, an antiviral agent.

24. A kit for diagnosing, preventing and/or treating an infection with a virus of the Flaviviridae family, comprising:
at least one inhibitor or antagonist of PADI4, PPARδ, GCKR and/or P2X4R,
and/or at least one nucleic acid or protein of PADI4, PPARδ, GCKR and/or P2X4R, optionally, suitable substrates of PADI4, PPARδ, GCKR and/or P2X4R for in vitro enzymatic assays,
optionally, a PADI4, PPARδ, GCKR and/or P2X4R test system; and
optionally, excipient(s) and further compounds.

25. The method of claim 22, wherein said method comprises:
(a) adding a compound to be screened to a PADI4, PPARδ, GCKR and/or P2X4R test system;
(b) infecting said test system with a virus of the Flaviviridae family in the presence of said compound to be screened;
(c) removing the viral inoculum and adding said compound to be screened;
(d) quantifying virus production; and
(e) comparing virus production in step (d) with virus production in the absence of the candidate compound, wherein a difference between the measured virus productions indicates that the candidate compound is a modulator of PADI4, PPARδ, GCKR and/or P2X4R, and wherein a decrease in the measured virus production in step (d) indicates that the candidate compound is an inhibitor or antagonist of PADI4, PPARδ, GCKR and/or P2X4R and is, thus, an antiviral agent.

26. The method of claim 22, wherein the test system is a cell line expressing PADI4, PPARδ, GCKR and/or P2X4R.


SEQUENCE LISTING (27 sequences)

SEQ ID NO: 1 - DNA, 2265 bp, Homo sapiens
acagccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtac

SEQ ID NO: 2 - protein, 663 aa, Homo sapiens
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 3 - DNA, 3749 bp, Homo sapiens
gcggagcgtgtgacgctgcggccgccgcggacctggggattaatgggaaaagttttggcaggagcgggagaattctgcggagcctgcgggacggcggcggtggcgccgtaggcagccgggacagtgttgtacagtgttttgggcatgcacgtgatactcacacagtggcttctgctcaccaacagatgaagacagatgcaccaacgaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagcccttgagcacccggcctggagcagcagagtcccacgatcgccctcagacacatgacacccacggcctctggctccctgtgccctctctcccgcttcctccagccagctctcttcctgtctttgttgtctccctctttctcagttcctctttcttttctaattcctgttgctctgtttcttcctttctgtaggtttctctcttcccttctcccttgccctccctttctctctccaccccccacgtctgtcctcctttcttattctgtgagatgttttgtattatttcaccagcagcatagaacaggacctctgcttttgcacaccttttccccaggagcagaagagagtggggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtgggcaggaggccccaggctgagagccagatgcctccccaagactgtcattgcccctccgatgctgaggccacccactgacccaactgatcctgctccagcagcacacctcagccccactgacacccagtgtccttccatcttcacactggtttgccaggccaatgttgctgatggccccctgcactggccgctggacggcactctcccagcttggaagtaggcagggttccctccaggtgggcccccacctcactgaagaggagcaagtctcaagagaaggaggggggattggtggttggaggaagcagcacacccaattctgcccctaggactcggggtctgagtcctggggtcaggccagggagagctcggggcaggccttccgccagcactcccactgcccccctgcccagtagcagccgcccacattgtgtcagcatccagggccagggcctggcctcacatccccctgctcctttctctagctggctccacgggagttcaggccccactccccctgaagctgcccctccagcacacacacataagcactgaaatcactttacctgcaggctccatgcacctcccttccctccctgaggcaggtgagaacccagagagaggggcctgcaggtgagcaggcagggctgggccaggtctccggggaggcaggggtcctgcaggtcctggtgggtcagcccagcacctgctcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctgtgtgtgactctgggtggaagtgcccagcccctgcccctacgggcgctgcagcctcccttccatgccccaggatcactctctgctggcaggattcttcccgctccccacctacccagctgatgggggttggggtgcttcctttcaggccaaggctatgaagggacagctgctgggacccacctccccctccccggccacatgccgcgtccctgccccgacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaagagctgactggaaaa

SEQ ID NO: 4 - protein, 441 aa, Homo sapiens
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 5 - DNA, 2028 bp, Homo sapiens
gcggagcgtgtgacgctgcggccgccgcggacctggggattaatgggaaaagttttggcaggagcgggagaattctgcggagcctgcgggacggcggcggtggcgccgtaggcagccgggacagtgttgtacagtgttttgggcatgcacgtgatactcacacagtggcttctgctcaccaacagatgaagacagatgcaccaacgaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggaggtgagtgagagtggggcaggtgggctggcctggcacacccagtcgtcctgggggttggccctcactgcagggcactgtgcctgagctctgacagtgtggggaagtgtccctgtgatcttggcagtggaacatgcaaggcactgactgagcatgcaggatcagctccatctcattatgtacgtagatagaggtggagacaggaaaaagactaagccagacgtggtggctcacacctgtaatcccagcactttggcaggccgaggcgggtggatcacttgaggtcaggagttcgaaaccagcctggccaacatggtgaaaccccgtctctactaaaaatacaaaaaattagccagatgtggtggcacgcgcctgtaatcccagctacttgggaggctgagccaggagaatcgcttgaacccgagaggtggaggttgcagtgagccaaaatcccaccactgcactccagcctgggtgacagagtgagaccctgtctcaaaaaaaaggaaaaggactaacaggcagtatgctgtcatgttaatgtggggtggaaaaattgtctgcattttttctgcatttttaaaattccaacacaataaatacaataataactatgctaaaaaaaaaaaaaaaaaaaaaaaaaa

SEQ ID NO: 6 - protein, 361 aa, Homo sapiens
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGGE

SEQ ID NO: 7 - DNA, 3518 bp, Homo sapiens
gcggagcgtgtgacgctgcggccgccgcggacctggggattaatgggaaaagttttggcaggagcgggagaattctgcggagcctgcgggacggcggcggtggcgccgtaggcagccgggacagtgttgtacagtgttttgggcatgcacgtgatactcacacagtggcttctgctcaccaacagatgaagacagatgcaccaacgagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagcccttgagcacccggcctggagcagcagagtcccacgatcgccctcagacacatgacacccacggcctctggctccctgtgccctctctcccgcttcctccagccagctctcttcctgtctttgttgtctccctctttctcagttcctctttcttttctaattcctgttgctctgtttcttcctttctgtaggtttctctcttcccttctcccttgccctccctttctctctccaccccccacgtctgtcctcctttcttattctgtgagatgttttgtattatttcaccagcagcatagaacaggacctctgcttttgcacaccttttccccaggagcagaagagagtggggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtgggcaggaggccccaggctgagagccagatgcctccccaagactgtcattgcccctccgatgctgaggccacccactgacccaactgatcctgctccagcagcacacctcagccccactgacacccagtgtccttccatcttcacactggtttgccaggccaatgttgctgatggccccctgcactggccgctggacggcactctcccagcttggaagtaggcagggttccctccaggtgggcccccacctcactgaagaggagcaagtctcaagagaaggaggggggattggtggttggaggaagcagcacacccaattctgcccctaggactcggggtctgagtcctggggtcaggccagggagagctcggggcaggccttccgccagcactcccactgcccccctgcccagtagcagccgcccacattgtgtcagcatccagggccagggcctggcctcacatccccctgctcctttctctagctggctccacgggagttcaggccccactccccctgaagctgcccctccagcacacacacataagcactgaaatcactttacctgcaggctccatgcacctcccttccctccctgaggcaggtgagaacccagagagaggggcctgcaggtgagcaggcagggctgggccaggtctccggggaggcaggggtcctgcaggtcctggtgggtcagcccagcacctgctcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctgtgtgtgactctgggtggaagtgcccagcccctgcccctacgggcgctgcagcctcccttccatgccccaggatcactctctgctggcaggattcttcccgctccccacctacccagctgatgggggttggggtgcttcctttcaggccaaggctatgaagggacagctgctgggacccacctccccctccccggccacatgccgcgtccctgccccgacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaagagctgactggaaaa

SEQ ID NO: 8 - protein, 402 aa, Homo sapiens
MHQRDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 9 - DNA, 3455 bp, Homo sapiens
gcggagcgtgtgacgctgcggccgccgcggacctggggattaatgggaaaagttttggcaggagcgggagaattctgcggagcctgcgggacggcggcggtggcgccgtaggcagccgggacagtgttgtacagtgttttgggcatgcacgtgatactcacacagtggcttctgctcaccaacagatgaagacagatgcaccaacgaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagcccttgagcacccggcctggagcagcagagtcccacgatcgccctcagacacatgacacccacggcctctggctccctgtgccctctctcccgcttcctccagccagctctcttcctgtctttgttgtctccctctttctcagttcctctttcttttctaattcctgttgctctgtttcttcctttctgtaggtttctctcttcccttctcccttgccctccctttctctctccaccccccacgtctgtcctcctttcttattctgtgagatgttttgtattatttcaccagcagcatagaacaggacctctgcttttgcacaccttttccccaggagcagaagagagtggggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtgggcaggaggccccaggctgagagccagatgcctccccaagactgtcattgcccctccgatgctgaggccacccactgacccaactgatcctgctccagcagcacacctcagccccactgacacccagtgtccttccatcttcacactggtttgccaggccaatgttgctgatggccccctgcactggccgctggacggcactctcccagcttggaagtaggcagggttccctccaggtgggcccccacctcactgaagaggagcaagtctcaagagaaggaggggggattggtggttggaggaagcagcacacccaattctgcccctaggactcggggtctgagtcctggggtcaggccagggagagctcggggcaggccttccgccagcactcccactgcccccctgcccagtagcagccgcccacattgtgtcagcatccagggccagggcctggcctcacatccccctgctcctttctctagctggctccacgggagttcaggccccactccccctgaagctgcccctccagcacacacacataagcactgaaatcactttacctgcaggctccatgcacctcccttccctccctgaggcaggtgagaacccagagagaggggcctgcaggtgagcaggcagggctgggccaggtctccggggaggcaggggtcctgcaggtcctggtgggtcagcccagcacctgctcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctgtgtgtgactctgggtggaagtgcccagcccctgcccctacgggcgctgcagcctcccttccatgccccaggatcactctctgctggcaggattcttcccgctccccacctacccagctgatgggggttggggtgcttcctttcaggccaaggctatgaagggacagctgctgggacccacctccccctccccggccacatgccgcgtccctgccccgacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaagagctgactggaaaa

SEQ ID NO: 10 - protein, 343 aa, Homo sapiens
MEQPQEEAPEVREEEEKEEVAEAEGAPELNGGPQHALPSSSYTAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 11 - DNA, 2197 bp, Homo sapiens
attgtgaccagaggggtttgtgtggctgaagaggcaggaggaacagtgtatccacagcgtgggaccatgccaggcacaaaacggtttcaacatgtcattgagaccccggagcctggcaagtgggagttgtctgggtacgaggcagctgtgccaatcacggagaagtcaaacccactgacccaggatctagacaaagcagatgctgagaacattgttcgactgctagggcaatgtgatgctgagatcttccaggaggaggggcaagccctgtccacataccagagactctacagcgaatccattctgaccaccatggtacaggtggctgggaaagttcaggaagtgctgaaggagccagatggggggctggttgtgctgagtggagggggcacctctggccggatggcattcctcatgtcggtgtcctttaatcagctgatgaaaggtctgggacagaaacctctttacacctacctcattgcaggtggtgacaggtctgtggtggcctctagggaggggacagaagatagtgccttgcacgggattgaggaactgaagaaggtggctgccgggaagaagagagtgattgtcattggcatttctgtgggactctctgctccctttgtggcaggccagatggactgctgcatgaacaacacagctgtcttcttgccagtcctggttggcttcaatccagtgagcatggccagaaatgaccccattgaagactggagttcaacattccgacaagtagcagagcggatgcagaaaatgcaggagaaacagaaagcttttgtgctcaatcctgccatcgggcccgagggtctcagcggctcctcccggatgaaaggtggaagtgccaccaagattctgctggaaaccctgttattagcagcccataagactgtggaccagggcattgcagcatctcaaagatgcctcctggaaatcttgcggacatttgagcgagctcatcaggtgacctacagccaaagccccaagattgccaccctgatgaagagtgtcagcaccagtctggagaagaaaggccacgtgtacctggttggctggcagaccctgggcatcattgccatcatggatggagtagagtgcatccacacctttggtgctgatttccgagatgtccgtggctttctcattggtgatcacagtgacatgtttaaccagaaggctgagctcaccaaccagggtccccagttcaccttctcccaggaggacttcctgacttccatccttccctctctcacggaaatcgatactgtggtcttcattttcaccctggatgacaacctcacggaggtgcagactatagtggagcaggtgaaagagaagaccaaccacatccaggccctggcacacagcaccgtgggtcagaccttgctgatccctctgaagaagctctttccctccatcatcagcatcacatggccactgcttttctttgaatatgaagggaacttcatccagaagttccagcgtgagctaagcaccaaatgggtgctgaatacagtgagtacaggtgctcatgtgcttcttggtaagatcctacaaaaccacatgttggaccttcggattagcaactccaagctcttctggcgggcgctggccatgctgcagcggttctctggacagtccaaggctcgatgcatcgagagcctcctccgagcgatccactttccccagccactgtcagatgatattcgggctgctcccatctcctgccatgtccaggttgcacatgagaaggaacaggtgatacccatcgccttgctgagcctcctattccggtgctcgatcactgaggctcaggcacacctggctgcagctccttctgtctgtgaggctgtcaggagtgctcttgctgggccaggtcagaagcgcactgcggaccccctcgagatcctagagcctgacgttcagtgaacccatgtttctgggtgggtgaaaggggcccaaccctgcccacttcagcccagcccgcccaaggggacttgtgccagcagaacatgtgggaggaagaagccccgtttccagggcatccgcagcccagggtagggagaaatattctctccactttgggggagagttcttgctctcgacctagtggtttctactctcaccgacttattctgatttcagaaataaaatgaaatgtcttattttggaaaaaaaaaaa

SEQ ID NO: 12 - protein, 625 aa, Homo sapiens
MPGTKRFQHVIETPEPGKWELSGYEAAVPITEKSNPLTQDLDKADAENIVRLLGQCDAEIFQEEGQALSTYQRLYSESILTTMVQVAGKVQEVLKEPDGGLVVLSGGGTSGRMAFLMSVSFNQLMKGLGQKPLYTYLIAGGDRSVVASREGTEDSALHGIEELKKVAAGKKRVIVIGISVGLSAPFVAGQMDCCMNNTAVFLPVLVGFNPVSMARNDPIEDWSSTFRQVAERMQKMQEKQKAFVLNPAIGPEGLSGSSRMKGGSATKILLETLLLAAHKTVDQGIAASQRCLLEILRTFERAHQVTYSQSPKIATLMKSVSTSLEKKGHVYLVGWQTLGIIAIMDGVECIHTFGADFRDVRGFLIGDHSDMFNQKAELTNQGPQFTFSQEDFLTSILPSLTEIDTVVFIFTLDDNLTEVQTIVEQVKEKTNHIQALAHSTVGQTLLIPLKKLFPSIISITWPLLFFEYEGNFIQKFQRELSTKWVLNTVSTGAHVLLGKILQNHMLDLRISNSKLFWRALAMLQRFSGQSKARCIESLLRAIHFPQPLSDDIRAAPISCHVQVAHEKEQVIPIALLSLLFRCSITEAQAHLAAAPSVCEAVRSALAGPGQKRTADPLEILEPDVQ

SEQ ID NO: 13 - DNA, 2091 bp, Homo sapiens
aagtgctgggatgacaggtgtgagccaccgcccccggcccctcgcccgccttttgaaggagcctttcgtcctcaagggcgaggccactccccccccgcgagttccatgccccctagagggtcatcgttcccgacggggaggtggcgccctcccccgggccccgggccccgaccgcccgtgctgcctccttccgggccctcctccgcgatgacggcgccgccagcaggccaggcggactgggcggggctccgagcggggactgggacccagaccgactaggggactgggagcgggcggcgcggccatggcgggctgctgcgccgcgctggcggccttcctgttcgagtacgacacgccgcgcatcgtgctcatccgcagccgcaaagtggggctcatgaaccgcgccgtgcaactgctcatcctggcctacgtcatcgggtgctaccatccccatttggcagaagtggaaatggagtcccctagaaggtgggtgtttgtgtgggaaaagggctaccaggaaactgactccgtggtcagctccgttacgaccaaggtcaagggcgtggctgtgaccaacacttctaaacttggattccggatctgggatgtggcggattatgtgataccagctcaggaggaaaactccctcttcgtcatgaccaacgtgatcctcaccatgaaccagacacagggcctgtgccccgagattccagatgcgaccactgtgtgtaaatcagatgccagctgtactgccggctctgccggcacccacagcaacggagtctcaacaggcaggtgcgtagctttcaacgggtctgtcaagacgtgtgaggtggcggcctggtgcccggtggaggatgacacacacgtgccacaacctgcttttttaaaggctgcagaaaacttcactcttttggttaagaacaacatctggtatcccaaatttaatttcagcaagaggaatatccttcccaacatcaccactacttacctcaagtcgtgcatttatgatgctaaaacagatcccttctgccccatattccgtcttggcaaaatagtggagaacgcaggacacagtttccaggacatggccgtggagggaggcatcatgggcatccaggtcaactgggactgcaacctggacagagccgcctccctctgcttgcccaggtactccttccgccgcctcgatacacgggacgttgagcacaacgtatctcctggctacaatttcaggtttgccaagtactacagagacctggctggcaacgagcagcgcacgctcatcaaggcctatggcatccgcttcgacatcattgtgtttgggaaggcagggaaatttgacatcatccccactatgatcaacatcggctctggcctggcactgctaggcatggcgaccgtgctgtgtgacatcatagtcctctactgcatgaagaaaagactctactatcgggagaagaaatataaatatgtggaagattacgagcagggtcttgctagtgagctggaccagtgaggcctaccccacacctgggctctccacagccccatcaaagaacagagaggaggaggagggagaaatggccaccacatcaccccagagaaatttctggaatctgattgagtctccactccacaagcactcagggttccccagcagctcctgtgtgttgtgtgcaggatctgtttgcccactcggcccaggaggtcagcagtctgttcttggctgggtcaactctgcttttcccgcaacctggggttgtcgggggagcgctggcccgacgcagtggcactgctgtggctttcagggctggagctggctttgctcagaagcctcctgtctccagctctctccaggacaggcccagtcctctgaggcacggcggctctgttcaagcactttatgcggcaggggaggccgcctggctgcagtcactagacttgtagcaggcctgggctgcaggcttccccccgaccattccctgcagccatgcggcagagctggcatttctcctcagagaagcgctgtgctaaggtgatcgaggaccagacattaaagcgtgattttcttaaaaaaaaaaaaaaaaa

SEQ ID NO: 14 - protein, 404 aa, Homo sapiens
MAGCCAALAAFLFEYDTPRIVLIRSRKVGLMNRAVQLLILAYVIGCYHPHLAEVEMESPRRWVFVWEKGYQETDSVVSSVTTKVKGVAVTNTSKLGFRIWDVADYVIPAQEENSLFVMTNVILTMNQTQGLCPEIPDATTVCKSDASCTAGSAGTHSNGVSTGRCVAFNGSVKTCEVAAWCPVEDDTHVPQPAFLKAAENFTLLVKNNIWYPKFNFSKRNILPNITTTYLKSCIYDAKTDPFCPIFRLGKIVENAGHSFQDMAVEGGIMGIQVNWDCNLDRAASLCLPRYSFRRLDTRDVEHNVSPGYNFRFAKYYRDLAGNEQRTLIKAYGIRFDIIVFGKAGKFDIIPTMINIGSGLALLGMATVLCDIIVLYCMKKRLYYREKKYKYVEDYEQGLASELDQ

SEQ ID NO: 15 - DNA, 2043 bp, Homo sapiens
aagtgctgggatgacaggtgtgagccaccgcccccggcccctcgcccgccttttgaaggagcctttcgtcctcaagggcgaggccactccccccccgcgagttccatgccccctagagggtcatcgttcccgacggggaggtggcgccctcccccgggccccgggccccgaccgcccgtgctgcctccttccgggccctcctccgcgatgacggcgccgccagcaggccaggcggactgggcggggctccgagcggggactgggacccagaccgactaggggactgggagcgggcggcgcggccatggcgggctgctgcgccgcgctggcggccttcctgttcgagtacgacacgccgcgcatcgtgctcatccgcagccgcaaagtggggctcatgaaccgcgccgtgcaactgctcatcctggcctacgtcatcgggtgggtgtttgtgtgggaaaagggctaccaggaaactgactccgtggtcagctccgttacgaccaaggtcaagggcgtggctgtgaccaacacttctaaacttggattccggatctgggatgtggcggattatgtgataccagctcaggaggaaaactccctcttcgtcatgaccaacgtgatcctcaccatgaaccagacacagggcctgtgccccgagattccagatgcgaccactgtgtgtaaatcagatgccagctgtactgccggctctgccggcacccacagcaacggagtctcaacaggcaggtgcgtagctttcaacgggtctgtcaagacgtgtgaggtggcggcctggtgcccggtggaggatgacacacacgtgccacaacctgcttttttaaaggctgcagaaaacttcactcttttggttaagaacaacatctggtatcccaaatttaatttcagcaagaggaatatccttcccaacatcaccactacttacctcaagtcgtgcatttatgatgctaaaacagatcccttctgccccatattccgtcttggcaaaatagtggagaacgcaggacacagtttccaggacatggccgtggagggaggcatcatgggcatccaggtcaactgggactgcaacctggacagagccgcctccctctgcttgcccaggtactccttccgccgcctcgatacacgggacgttgagcacaacgtatctcctggctacaatttcaggtttgccaagtactacagagacctggctggcaacgagcagcgcacgctcatcaaggcctatggcatccgcttcgacatcattgtgtttgggaaggcagggaaatttgacatcatccccactatgatcaacatcggctctggcctggcactgctaggcatggcgaccgtgctgtgtgacatcatagtcctctactgcatgaagaaaagactctactatcgggagaagaaatataaatatgtggaagattacgagcagggtcttgctagtgagctggaccagtgaggcctaccccacacctgggctctccacagccccatcaaagaacagagaggaggaggagggagaaatggccaccacatcaccccagagaaatttctggaatctgattgagtctccactccacaagcactcagggttccccagcagctcctgtgtgttgtgtgcaggatctgtttgcccactcggcccaggaggtcagcagtctgttcttggctgggtcaactctgcttttcccgcaacctggggttgtcgggggagcgctggcccgacgcagtggcactgctgtggctttcagggctggagctggctttgctcagaagcctcctgtctccagctctctccaggacaggcccagtcctctgaggcacggcggctctgttcaagcactttatgcggcaggggaggccgcctggctgcagtcactagacttgtagcaggcctgggctgcaggcttccccccgaccattccctgcagccatgcggcagagctggcatttctcctcagagaagcgctgtgctaaggtgatcgaggaccagacattaaagcgtgattttcttaaaaaaaaaaaaaaaaa

SEQ ID NO: 16 - protein, 388 aa, Homo sapiens
MAGCCAALAAFLFEYDTPRIVLIRSRKVGLMNRAVQLLILAYVIGWVFVWEKGYQETDSVVSSVTTKVKGVAVTNTSKLGFRIWDVADYVIPAQEENSLFVMTNVILTMNQTQGLCPEIPDATTVCKSDASCTAGSAGTHSNGVSTGRCVAFNGSVKTCEVAAWCPVEDDTHVPQPAFLKAAENFTLLVKNNIWYPKFNFSKRNILPNITTTYLKSCIYDAKTDPFCPIFRLGKIVENAGHSFQDMAVEGGIMGIQVNWDCNLDRAASLCLPRYSFRRLDTRDVEHNVSPGYNFRFAKYYRDLAGNEQRTLIKAYGIRFDIIVFGKAGKFDIIPTMINIGSGLALLGMATVLCDIIVLYCMKKRLYYREKKYKYVEDYEQGLASELDQ

SEQ ID NO: 17 - DNA, 2023 bp, Homo sapiens
aagtgctgggatgacaggtgtgagccaccgcccccggcccctcgcccgccttttgaaggagcctttcgtcctcaagggcgaggccactccccccccgcgagttccatgccccctagagggtcatcgttcccgacggggaggtggcgccctcccccgggccccgggccccgaccgcccgtgctgcctccttccgggccctcctccgcgatgacggcgccgccagcaggccaggcggactgggcggggctccgagcggggactgggacccagaccgactaggggactgggagcgggcggcgcggccatggcgggctgctgcgccgcgctggcggccttcctgttcgagtacgacacgccgcgcatcgtgctcatccgcagccgcaaagtggggctcatgaaccgcgccgtgcaactgctcatcctggcctacgtcatcgggtgggtgtttgtgtgggaaaagggctaccaggaaactgactccgtggtcagctccgttacgaccaaggtcaagggcgtggctgtgaccaacacttctaaacttggattccggatctgggatgtggcggattatgtgataccagctcaggaggaaaactccctcttcgtcatgaccaacgtgatcctcaccatgaaccagacacagggcctgtgccccgagattccagatgcgaccactgtgtgtaaatcagatgccagctgtactgccggctctgccggcacccacagcaacggagtctcaacaggcagacctgcttttttaaaggctgcagaaaacttcactcttttggttaagaacaacatctggtatcccaaatttaatttcagcaagaggaatatccttcccaacatcaccactacttacctcaagtcgtgcatttatgatgctaaaacagatcccttctgccccatattccgtcttggcaaaatagtggagaacgcaggacacagtttccaggacatggccgtggagggaggcatcatgggcatccaggtcaactgggactgcaacctggacagagccgcctccctctgcttgcccaggtactccttccgccgcctcgatacacgggacgttgagcacaacgtatctcctggctacaatttcaggtttgccaagtactacagagacctggctggcaacgagcagcgcacgctcatcaaggcctatggcatccgcttcgacatcattgtgtttgggaaggcagggaaatttgacatcatccccactatgatcaacatcggctctggcctggcactgctaggcatggcgaccgtgctgtgtgacatcatagtcctctactgcatgaagaaaagactctactatcgggagaagaaatataaatatgtggaagattacgagcagggtcttgctagtgagctggaccagtgaggcctaccccacacctgggctctccacagccccatcaaagaacagagaggaggaggagggagaaatggccaccacatcaccccagagaaatttctggaatctgattgagtctccactccacaagcactcagggttccccagcagctcctgtgtgttgtgtgcaggatctgtttgcccactcggcccaggaggtcagcagtctgttcttggctgggtcaactctgcttttcccgcaacctggggttgtcgggggagcgctggcccgacgcagtggcactgctgtggctttcagggctggagctggctttgctcagaagcctcctgtctccagctctctccaggacaggcccagtcctctgaggcacggcggctctgttcaagcactttatgcggcaggggaggccgcctggctgcagtcactagacttgtagcaggcctgggctgcaggcttccccccgaccattccctgcagccatgcggcagagctggcatttctcctcagagaagcgctgtgctaaggtgatcgaggaccagacattaaagcgtgattttcttaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - protein, 361 aa, Homo sapiens
MAGCCAALAAFLFEYDTPRIVLIRSRKVGLMNRAVQLLILAYVIGWVFVWEKGYQETDSVVSSVTTKVKGVAVTNTSKLGFRIWDVADYVIPAQEENSLFVMTNVILTMNQTQGLCPEIPDATTVCKSDASCTAGSAGTHSNGVSTGRPAFLKAAENFTLLVKNNIWYPKFNFSKRNILPNITTTYLKSCIYDAKTDPFCPIFRLGKIVENAGHSFQDMAVEGGIMGIQVNWDCNLDRAASLCLPRYSFRRLDTRDVEHNVSPGYNFRFAKYYRDLAGNEQRTLIKAYGIRFDIIVFGKAGKFDIIPTMINIGSGLALLGMATVLCDIIVLYCMKKRLYYREKKYKYVEDYEQGLASELDQ

SEQ ID NO: 19 - DNA, 1796 bp, Homo sapiens
aagtgctgggatgacaggtgtgagccaccgcccccggcccctcgcccgccttttgaaggagcctttcgtcctcaagggcgaggccactccccccccgcgagttccatgccccctagagggtcatcgttcccgacggggaggtggcgccctcccccgggccccgggccccgaccgcccgtgctgcctccttccgggccctcctccgcgatgacggcgccgccagcaggccaggcggactgggcggggctccgagcggggactgggacccagaccgactaggggactgggagcgggcggcgcggccatggcgggctgctgcgccgcgctggcggccttcctgttcgagtacgacacgccgcgcatcgtgctcatccgcagccgcaaagtggggctcatgaaccgcgccgtgcaactgctcatcctggcctacgtcatcgggtgggtgtttgtgtgggaaaagggctaccaggaaactgactccgtggtcagctccgttacgaccaaggtcaagggcgtggctgtgaccaacacttctaaacttggattccggatctgggatgtggcggattatgtgataccagctcaggaggaaaactccctcttcgtcatgaccaacgtgatcctcaccatgaaccagacacagggcctgtgccccgagattccagatgcgaccactgtgtgtaaatcagatgccagctgtactgccggctctgccggcacccacagcaacggagtctcaacaggcaggtgcgtagctttcaacgggtctgtcaagacgtgtgaggtggcggcctggtgcccggtggaggatgacacacacgtgccacaacctgcttttttaaaggctgcagaaaacttcactcttttggttaagaacaacatctggtatcccaaatttaatttcagcaagaggaatatccttcccaacatcaccactacttacctcaagtcgtgcatttatgatgctaaaacagatcccttctgccccatattccgtcttggcaaaatagtggagaacgcaggacacagtttccaggacatggccgtggagggaggcatcatgggcatccaggtcaactgggactgcaacctggacagagccgcctccctctgcttgcccaggtactccttccgccgcctcgatacacgggacgttgagcacaacgtatctcctggctacaatttcaggtttgccaagtactacagagacctggctggcaacgagcagcgcacgctcatcaaggcctatggcatccgcttcgacatcattgtgtttgggaaggcagggaaatttgacatcaccccagagaaatttctggaatctgattgagtctccactccacaagcactcagggttccccagcagctcctgtgtgttgtgtgcaggatctgtttgcccactcggcccaggaggtcagcagtctgttcttggctgggtcaactctgcttttcccgcaacctggggttgtcgggggagcgctggcccgacgcagtggcactgctgtggctttcagggctggagctggctttgctcagaagcctcctgtctccagctctctccaggacaggcccagtcctctgaggcacggcggctctgttcaagcactttatgcggcaggggaggccgcctggctgcagtcactagacttgtagcaggcctgggctgcaggcttccccccgaccattccctgcagccatgcggcagagctggcatttctcctcagagaagcgctgtgctaaggtgatcgaggaccagacattaaagcgtgattttcttaaaaaaaaaaaaaaaaaaa

SEQ ID NO: 20 - protein, 341 aa, Homo sapiens
MAGCCAALAAFLFEYDTPRIVLIRSRKVGLMNRAVQLLILAYVIGWVFVWEKGYQETDSVVSSVTTKVKGVAVTNTSKLGFRIWDVADYVIPAQEENSLFVMTNVILTMNQTQGLCPEIPDATTVCKSDASCTAGSAGTHSNGVSTGRCVAFNGSVKTCEVAAWCPVEDDTHVPQPAFLKAAENFTLLVKNNIWYPKFNFSKRNILPNITTTYLKSCIYDAKTDPFCPIFRLGKIVENAGHSFQDMAVEGGIMGIQVNWDCNLDRAASLCLPRYSFRRLDTRDVEHNVSPGYNFRFAKYYRDLAGNEQRTLIKAYGIRFDIIVFGKAGKFDITPEKFLESD

SEQ ID NO: 21 - DNA, 57 bp, Artificial Sequence
  control shRNA shNT#1
ccggcaacaagatgaagagcaccaactcgagttggtgctcttcatcttgttgttttt

SEQ ID NO: 22 - DNA, 57 bp, Artificial Sequence; identical to SEQ ID NO: 21
  control shRNA shNT#2

SEQ ID NO: 23 - DNA, 58 bp, Artificial Sequence
  shRNA shPADI4#1
ccggctgaaggagtttcccatcaaactcgagtttgatgggaaactccttcagtttttg

SEQ ID NO: 24 - DNA, 59 bp, Artificial Sequence
  shRNA shPADI4#2
ccggagacattgagagaacataattctcgagaattatgttctctcaatgtctttttttg

SEQ ID NO: 25 - DNA, 59 bp, Artificial Sequence
  shRNA shPADI4#3
ccggtgactactctggccatgaaagctcgagctttcatggccagagtagtcattttttg

SEQ ID NO: 26 - DNA, 59 bp, Artificial Sequence
  shRNA shPADI4#4
ccggccaggtctgagatggacaaagctcgagctttgtccatctcagacctggttttttg

SEQ ID NO: 27 - DNA, 59 bp, Artificial Sequence
  shRNA shPADI4#5
ccggagcaagagctcttgtgaatatctcgagatattcacaagagctcttgctttttttg